(19) United States
(12) Patent Application Publication
(10) Pub. No.: US 2025/0134936 A1
(43) Pub. Date: May 1, 2025

(54) METHODS AND COMPOSITIONS FOR AQUACULTURE

(71) Applicant: Prospective Research, Inc., Beverly, MA (US)

(72) Inventors: Dakota Hamill, Beverly, MA (US); Neal Connors, Beverly, MA (US)

(73) Assignee: Prospective Research, Inc., Beverly, MA (US)

(21) Appl. No.: 18/682,564

(22) PCT Filed: Aug. 9, 2022
(86) PCT No.: PCT/US2022/039795

§ 371 Date: Feb. 9, 2024

Related U.S. Application Data
(60) Provisional application No. 63/233,123, filed on Aug. 13, 2021.

(51) Int. Cl. A61K 35/742; A23K 10/18; A23K 50/80; C12N 1/20; C12R 1/01

(52) U.S. Cl. CPC A61K 35/742; A23K 10/18; A23K 50/80; C12N 1/205; C12R 2001/01

(57) ABSTRACT

Disclosed herein are compositions and methods for preventing, ameliorating, or treating pathogenic infections in aquaculture and/or reducing the severity of one or more risk factors, signs, or symptoms associated with pathogenic infections in aquaculture. In particular, the technology of the present disclosure relates to methods for administering an effective amount of a composition comprising a Paenibacillus strain, identified as PR-D9, to a subject suffering from or at risk for a pathogenic infection associated with aquaculture.

### CROSS-REFERENCE TO RELATED APPLICATION

The present application claims priority to U.S. Provisional Patent Application No. 63/233,123, filed on Aug. 13, 2021, the contents of which are hereby incorporated by reference in their entirety.

### TECHNICAL FIELD

The present technology relates to methods and compositions for preventing, ameliorating, or treating pathogenic infections in aquaculture and/or reducing the severity of one or more risk factors, signs, or symptoms associated with pathogenic infections in aquaculture. In particular, the present technology relates to administering an effective amount of a composition comprising a Paenibacillus strain, identified as PR-D9, to a subject suffering from or at risk for a pathogenic infection associated with aquaculture.

### BACKGROUND

The following description is provided to assist the understanding of the reader. None of the information provided or references cited is admitted to be prior art to the compositions and methods disclosed herein.

The aquaculture industry comprises cultured fish, crustaceans, and shellfish. This cluster of multi-billion-dollar industries contributes to food security, the supply of dense nutrition, and economic stimulus globally. Demand for cultured finfish and crustaceans increased 70% and 58%, respectively between 2010 and 2018. Pathogenic threats are however a barrier to growth and sustainability of these expanding industries. Breeding, nutrition, pharmacology, engineering, and disease management strategies are active areas of focus for minimizing losses attributed to these pathogenic threats.

A non-exhaustive list of problematic pathogens for the aquaculture industry includes: Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum. These pathogens account for billions of dollars of losses to these industries annually. For example, in Brazil alone, the 13th highest producing country of aquaculture species, USD 84 million in losses to pathogen infections was estimated for freshwater fish in 2020. An unpublished estimate from the World Bank suggests that greater than 3 billion in losses to the shrimp aquaculture industry alone are caused by disease.

Feed additives are a promising means to reduce the need for antibiotics and medicated feed use in aquaculture. This is a key area of demand within the aquaculture industry as improvements related to environmental impacts, public perception, and animal welfare are vital to sustainability. Some current feed additives include signaling molecules that modulate bacterial behavior by selectively activating bacterial genes encoding therapeutics inside the gut of fish, shrimp, and shellfish, which can protect against pathogen infections. These treatment types do not have the same drawbacks as drug and pesticide treatments and thus, are in high demand if similar levels of efficacy can be reached. Moreover, food additives that optimize nutrient absorption, such as by breaking down products for more optimal animal consumption, can add significant value and efficiency gains to aquaculture farms. Accordingly, there is a need for food additives that display a high degree of efficacy against a broad range of target pathogens, with minimal deleterious impacts on animal health and the environment, and optimally with improved nutrient availability for the farmed animal.

### SUMMARY

In one aspect, the disclosure of the present technology provides an isolated Paenibacillus strain PR-D9 deposited with the Agricultural Research Culture Collection (NRRL—Northern Regional Research Laboratory) under NRRL Accession Number B-68028 or a variant strain thereof. In some embodiments, the isolated Paenibacillus strain is a lyophilized bacterial fermentation (LBF). In some embodiments, the isolated Paenibacillus strain is spray dried. In some embodiments, the isolated Paenibacillus strain is heat-stable. In some embodiments, the isolated Paenibacillus strain is capable of reducing or inhibiting growth of one or more pathogens selected from the group consisting of Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum, or any combination thereof. In some embodiments, the isolated Paenibacillus strain is capable of inhibiting growth of one or more pathogens selected from the group consisting of Vibrio parahaemolyticus, Aeromonas salmonicida, Flavobacterium psychrophilium, Photobacterium damselae piscida, Vibrio anguillarum, Yersinia ruckeri, Streptococcus agalactiae, Streptococcus iniae, Saprolegnia parasitica, Tenacibaculum maritimum, Moritella viscosa, Flavobacterium columnare, and Renibacterium salmoninarum, at minimum inhibitory concentrations (MIC) of 0.78-0.025 mg/mL. In some embodiments, the isolated Paenibacillus strain, after having been subjected to a heat treatment to form a heat-treated Paenibacillus strain, is capable of reducing or inhibiting the growth of the one or more pathogens. In some embodiments, the isolated Paenibacillus strain is capable of reducing or inhibiting pathogen growth to an extent that is substantially the same as a control isolated Paenibacillus strain PR-D9 that was not subjected to heat treatment. In some embodiments, the pathogen is Vibrio parahaemolyticus. In some embodiments, the heat treatment comprises autoclaving. In some embodiments, the autoclaving is performed at a temperature of 121° C. and pressure of 15 psi for 15 minutes. In some embodiments, the strain is fed to an aquaculture animal, the strain reduces the risk of, prevents, or treats one or more conditions selected from the group consisting of acute hepatopancreatic necrosis disease (AHPND), early mortality syndrome (EMS), white feces disease (WFD), white spot syndrome, piscirickettiosis, photobacteriosis, meningitis, bacterial kidney disease (BKD), tenacibaculosis, white ulcer disease, columnaris disease, furunculosis, vibriosis, and bacterial cold water disease (BCWD) in the aquaculture animal as compared to control aquaculture animal. In some embodiments, when the strain is fed to an aquaculture animal, the strain increases one or more of the growth and survival of the aquaculture animal as compared to a control aquaculture animal. In some embodiments, the aquaculture animal is a crustacean or cultured fish. In some embodiments, the aquaculture animal is a crustacean. In some embodiments, the crustacean is shrimp. In some embodiments, the Paenibacillus strain produces exoenzymes that digest soy protein.

In some embodiments, the disclosure of the present technology relates to a food or feed additive comprising the isolated Paenibacillus strain. In some embodiments, the food is an aquaculture food and the feed additive is an aquaculture feed additive. In some embodiments, the aquaculture food comprises 0.25%, 0.5%, 1%, 5%, or 10% (w/w) Paenibacillus strain.

In some embodiments, the Paenibacillus strain is a lyophilized bacterial fermentation.

In one aspect, the disclosure of the present technology provides a composition comprising: (i) an isolated Paenibacillus strain PR-D9 deposited with the Agricultural Research Culture Collection (NRRL—Northern Regional Research Laboratory) under NRRL Accession Number B-68028 or a variant strain thereof; and (ii) an excipient. In some embodiments, the Paenibacillus strain is lyophilized. In some embodiments, the Paenibacillus strain is a lyophilized bacterial fermentation (LBF). In some embodiments, the Paenibacillus strain is spray dried. In some embodiments, the excipient comprises one or more lyoprotectants selected from the group consisting of mannitol, trehalose, sucrose, glycerol, glycine, skim milk, bovine serum albumin (BSA), and a lyophilization buffer, or any combination thereof. In some embodiments, the Paenibacillus strain is heat-stable. In some embodiments, the composition is capable of reducing or inhibiting growth of one or more pathogens selected from the group consisting of Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum, or any combination thereof. In some embodiments, the composition is capable of inhibiting growth of one or more pathogens selected from the group consisting of Vibrio parahaemolyticus, Aeromonas salmonicida, Flavobacterium psychrophilium, Photobacterium damselae piscida, Vibrio anguillarum, Yersinia ruckeri, Streptococcus agalactiae, Streptococcus iniae, Saprolegnia parasitica, Tenacibaculum maritimum, Moritella viscosa, Flavobacterium columnare, and Renibacterium salmoninarum, at minimum inhibitory concentrations (MIC) of 0.78-0.025 mg/mL. In some embodiments, the composition, after having been subjected to a heat treatment to form a heat-treated composition, is capable of reducing or inhibiting the growth of the one or more pathogens. In some embodiments, the heat-treated composition is capable of reducing or inhibiting pathogen growth to an extent that is substantially the same as a control composition that was not subjected to heat treatment. In some embodiments, the heat treatment comprises autoclaving. In some embodiments, the autoclaving is performed at a temperature of 121° C. and pressure of 15 psi for 15 minutes. In some embodiments, the composition is fed to an aquaculture animal, the strain reduces the risk of, prevents, or treats one or more conditions selected from the group consisting of acute hepatopancreatic necrosis disease (AHPND), early mortality syndrome (EMS), white feces disease (WFD), white spot syndrome, piscirickettiosis, photobacteriosis, meningitis, bacterial kidney disease (BKD), tenacibaculosis, white ulcer disease, columnaris disease, furunculosis, vibriosis, and bacterial cold water disease (BCWD) in the aquaculture animal. In some embodiments, when the composition is fed to an aquaculture animal, the composition increases one or more of the growth and survival of the aquaculture animal as compared to a control aquaculture animal. In some embodiments, the aquaculture animal is a crustacean or cultured fish. In some embodiments, the aquaculture animal is a crustacean. In some embodiments, the crustacean is shrimp. In some embodiments, the composition is formulated for oral administration. In some embodiments, the Paenibacillus strain produces exoenzymes that digest soy protein. In some embodiments, the composition is formulated as food or a feed additive. In some embodiments, the composition is formulated as an aquaculture food or an aquaculture feed additive. In some embodiments, the composition is formulated as an aquaculture food comprising 1%, 5%, or 10% (w/w) Paenibacillus strain. In some embodiments, the Paenibacillus strain is a lyophilized bacterial fermentation. In some embodiments, the composition is formulated as an aquaculture bath.

In one aspect, the disclosure of the present technology provides a method of reducing the risk of, or preventing, or treating an aquaculture pathogen infection in a subject in need thereof, comprising administering to the subject an isolated Paenibacillus strain PR-D9 deposited with the Agricultural Research Culture Collection (NRRL—Northern Regional Research Laboratory) under NRRL Accession Number B-68028 or a variant strain thereof. In some embodiments, the Paenibacillus strain is formulated as a composition, wherein the composition comprises: (i) the Paenibacillus strain; and (ii) an excipient. In some embodiments, the Paenibacillus strain is lyophilized. In some embodiments, the lyophilized Paenibacillus strain is a lyophilized bacterial fermentation (LBF). In some embodiments, the Paenibacillus strain is spray dried. In some embodiments, the excipient comprises one or more lyoprotectants selected from the group consisting of mannitol, trehalose, sucrose, glycerol, glycine, skim milk, bovine serum albumin (BSA), and a lyophilization buffer, or any combination thereof. In some embodiments, the Paenibacillus strain is heat-stable. In some embodiments, reducing the risk of, or preventing, or treating an aquaculture pathogen infection in an aquaculture environment comprises reducing or inhibiting growth of one or more pathogens selected from the group consisting of Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum, or any combination thereof. In some embodiments, the aquaculture pathogen infection comprises one or more conditions selected from the group consisting of acute hepatopancreatic necrosis disease (AHPND), early mortality syndrome (EMS), white feces disease (WFD), white spot syndrome, piscirickettiosis, photobacteriosis, meningitis, bacterial kidney disease (BKD), tenacibaculosis, white ulcer disease, columnaris disease, furunculosis, vibriosis, and bacterial cold water disease (BCWD). In some embodiments, the subject is an aquaculture animal. In some embodiments, the aquaculture animal is a crustacean or cultured fish. In some embodiments, the aquaculture animal is a crustacean. In some embodiments, the crustacean is shrimp. In some embodiments, the pathogen is Vibrio parahaemolyticus. In some embodiments, the aquaculture pathogen infection is acute hepatopancreatic necrosis disease (AHPND). In some embodiments, the Paenibacillus strain is administered orally to the subject. In some embodiments, the Paenibacillus strain is administered to the subject as food or a feed additive. In some embodiments, the Paenibacillus strain is administered to the subject as an aquaculture food or an aquaculture feed additive. In some embodiments, the aquaculture food comprises 1%, 5%, or 10% (w/w) Paenibacillus strain. In some embodiments, the Paenibacillus strain is a lyophilized bacterial fermentation.

In one aspect, the disclosure of the present technology provides a method for increasing the survival of an aquaculture animal exposed to an aquaculture pathogen, comprising administering to the subject an isolated Paenibacillus strain PR-D9 deposited with the Agricultural Research Culture Collection (NRRL—Northern Regional Research Laboratory) under NRRL Accession Number B-68028 or a variant strain thereof, wherein the survival of the aquaculture animal is increased as compared to an untreated control. In some embodiments, the Paenibacillus strain is formulated as a composition, wherein the composition comprises: (i) the Paenibacillus strain; and (ii) an excipient. In some embodiments, the Paenibacillus strain is lyophilized. In some embodiments, the lyophilized Paenibacillus strain is a lyophilized bacterial fermentation (LBF). In some embodiments, the Paenibacillus strain is spray dried. In some embodiments, the excipient comprises one or more lyoprotectants selected from the group consisting of mannitol, trehalose, sucrose, glycerol, glycine, skim milk, bovine serum albumin (BSA), and a lyophilization buffer, or any combination thereof. In some embodiments, the Paenibacillus strain is heat-stable. In some embodiments, reducing the risk of, or preventing, or treating an aquaculture pathogen infection in an aquaculture environment comprises reducing or inhibiting growth of one or more pathogens selected from the group consisting of Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum, or any combination thereof. In some embodiments, the aquaculture pathogen infection comprises one or more conditions selected from the group consisting of acute hepatopancreatic necrosis disease (AHPND), early mortality syndrome (EMS), white feces disease (WFD), white spot syndrome, piscirickettiosis, photobacteriosis, meningitis, bacterial kidney disease (BKD), tenacibaculosis, white ulcer disease, columnaris disease, furunculosis, vibriosis, and bacterial cold water disease (BCWD). In some embodiments, the subject is an aquaculture animal. In some embodiments, the aquaculture animal is a crustacean or cultured fish. In some embodiments, the aquaculture animal is a crustacean. In some embodiments, the crustacean is shrimp. In some embodiments, the pathogen is Vibrio parahaemolyticus. In some embodiments, the aquaculture pathogen infection is acute hepatopancreatic necrosis disease (AHPND). In some embodiments, the Paenibacillus strain is administered orally to the subject. In some embodiments, the Paenibacillus strain is administered to the subject as food or a feed additive. In some embodiments, the Paenibacillus strain is administered to the subject as an aquaculture food or an aquaculture feed additive. In some embodiments, the aquaculture food comprises 1%, 5%, or 10% (w/w) Paenibacillus strain. In some embodiments, the Paenibacillus strain is a lyophilized bacterial fermentation.

In one aspect, the disclosure of the present technology provides a method for improving the growth of an aquaculture animal, comprising administering to the subject an isolated Paenibacillus strain PR-D9 deposited with the Agricultural Research Culture Collection (NRRL—Northern Regional Research Laboratory) under NRRL Accession Number B-68028 or a variant strain thereof, wherein the growth of the aquaculture animal is increased as compared to an untreated control. the Paenibacillus strain is formulated as a composition, wherein the composition comprises: (i) the Paenibacillus strain; and (ii) an excipient. In some embodiments, the Paenibacillus strain is lyophilized. In some embodiments, the lyophilized Paenibacillus strain is a lyophilized bacterial fermentation (LBF). In some embodiments, the Paenibacillus strain is spray dried. In some embodiments, the excipient comprises one or more lyoprotectants selected from the group consisting of mannitol, trehalose, sucrose, glycerol, glycine, skim milk, bovine serum albumin (BSA), and a lyophilization buffer, or any combination thereof. In some embodiments, the Paenibacillus strain is heat-stable. In some embodiments, reducing the risk of, or preventing, or treating an aquaculture pathogen infection in an aquaculture environment comprises reducing or inhibiting growth of one or more pathogens selected from the group consisting of Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum, or any combination thereof. In some embodiments, the aquaculture pathogen infection comprises one or more conditions selected from the group consisting of acute hepatopancreatic necrosis disease (AHPND), early mortality syndrome (EMS), white feces disease (WFD), white spot syndrome, piscirickettiosis, photobacteriosis, meningitis, bacterial kidney disease (BKD), tenacibaculosis, white ulcer disease, columnaris disease, furunculosis, vibriosis, and bacterial cold water disease (BCWD). In some embodiments, the subject is an aquaculture animal. In some embodiments, the aquaculture animal is a crustacean or cultured fish. In some embodiments, the aquaculture animal is a crustacean. In some embodiments, the crustacean is shrimp. In some embodiments, the pathogen is Vibrio parahaemolyticus. In some embodiments, the aquaculture pathogen infection is acute hepatopancreatic necrosis disease (AHPND). In some embodiments, the Paenibacillus strain is administered orally to the subject. In some embodiments, the Paenibacillus strain is administered to the subject as food or a feed additive. In some embodiments, the Paenibacillus strain is administered to the subject as an aquaculture food or an aquaculture feed additive. In some embodiments, the aquaculture food comprises 1%, 5%, or 10% (w/w) Paenibacillus strain. In some embodiments, the Paenibacillus strain is a lyophilized bacterial fermentation.

In one aspect, the disclosure of the present technology provides a method for improving the nutritional availability of soy protein to an aquaculture animal, the method comprising administering an isolated Paenibacillus strain PR-D9 deposited with the Agricultural Research Culture Collection (NRRL—Northern Regional Research Laboratory) under NRRL Accession Number B-68028 or a variant strain thereof to the aquaculture animal. In some embodiments, the Paenibacillus strain is formulated as a composition, wherein the composition comprises: (i) the Paenibacillus strain; and (ii) an excipient. In some embodiments, the Paenibacillus strain is lyophilized. In some embodiments, the lyophilized Paenibacillus strain is a lyophilized bacterial fermentation (LBF). In some embodiments, the Paenibacillus strain is spray dried. In some embodiments, the excipient comprises one or more lyoprotectants selected from the group consisting of mannitol, trehalose, sucrose, glycerol, glycine, skim milk, bovine serum albumin (BSA), and a lyophilization buffer, or any combination thereof. In some embodiments, the Paenibacillus strain is heat-stable. In some embodiments, the isolated Paenibacillus strain is administered separately, sequentially, or simultaneously with an aquaculture food. In some embodiments, the isolated Paenibacillus strain is administered to the subject as an aquaculture food or an aquaculture feed additive.

### BRIEF DESCRIPTION OF THE DRAWINGS

FIG. 1A is a photo of an initial soil isolation plate of actinomycetes isolation agar (AIA), with an overlayed Vibrio parahaemolyticus ATCC 17802 pathogen. Large zone of inhibition present around a colony of strain of interest identified as PR-D9.

FIGS. 1B and 1C are photos of the top side (FIG. 1C) and reverse (FIG. 1B) side of the same agar plate displaying two treatments applied to a TSA+2-3% NaCl agar plate overlaid with a 0.005 OD culture of V. parahaemolyticus. Treatment (A) is 5 μL of whole PR-D9 fermentation, treatment (B) is 50 μL of cell-free supernatant from PR-D9 growth in a sterile well.

FIG. 1D is a photo of an agar plate of Mueller Hinton Agar overlayed with ATCC 17802 Vibrio parahaemolyticus with 5 μL of fermentation broth from various soil isolates of bacteria, internally named 3H3, 13G2, 13H7, and D9. Strain of interest, D9, shows zones of clearing against pathogen.

FIGS. 1E and 1F are plate images displaying a 5 μL treatment of PR-D9 fermentation broth applied to a Mueller Hinton agar plate overlaid with a culture of Streptococcus iniae (FIG. 1E) and Streptococcus agalactiae (FIG. 1F). In the center of each plate, clear zones of inhibition can be seen against the aquaculture pathogens.

FIGS. 1G and 1H are plate images displaying a 50 μL treatment of cell-free supernatant from PR-D9 growth applied to a sterile well bored into a Mueller Hinton agar plate overlaid with a culture of Streptococcus iniae (FIG. 1G) and Streptococcus agalactiae (FIG. 111). In the center of each plate, clear zones of inhibition can be seen against the aquaculture pathogens.

FIG. 2 is a boxplot displaying Delta OD₆₀₀ readings from liquid broth assay for each compound concentration (mg/mL) with V. parahaemolyticus. The dashed line indicates no change in OD₆₀₀ over the incubation period. Groups with a * symbol are significantly different from the positive control using Dunnett's Test (p<0.05). “LBF”=lyophilized bacterial fermentation.

FIG. 3A is a boxplot displaying Delta OD₆₀₀ readings from liquid broth assay for each compound concentration (mg/mL) with S. agalactiae. The dashed line indicates no change in OD₆₀₀ over the incubation period. Groups with a * symbol are significantly different from the positive control using Dunnett's Test (p<0.05). “LBF”=lyophilized bacterial fermentation.

FIG. 3B is a photo of plating of 100 μL from combined wells of S. agalactiae treated with PR-D9 (12.5 mg/mL).

FIG. 4A is a boxplot displaying Delta OD₆₀₀ readings from liquid broth assay for each compound concentration (mg/mL) with S. iniae. The dashed line indicates no change in OD₆₀₀ over the incubation period. Groups with a * symbol are significantly different from the positive control using Dunnett's Test (p<0.05). “LBF”=lyophilized bacterial fermentation.

FIG. 4B is a photo of plating of 100 μL from combined wells of S. iniae treated with PR-D9 (12.5 mg/mL).

FIG. 4C is a plate image displaying an S. iniae agar diffusion assay using PR-D9. Numbers represent the 1:2 dilution series, where dilution 1 is neat, dilution 2 is 1:2 dilution, dilution 3 is 1:4 dilution, etc.

FIG. 5A is a boxplot displaying Delta OD₆₀₀ readings from liquid broth assay for each compound concentration (mg/mL) with A. salmonicida. The dashed line indicates no change in OD₆₀₀ over the incubation period. Groups with a * symbol are significantly different from the positive control using Dunnett's Test (p<0.05). “LBF”=lyophilized bacterial fermentation.

FIG. 5B is a photo of plating of 100 μL from combined wells of A. salmonicida treated with PR-D9 (12.5 mg/mL).

FIG. 5C is a plate image displaying an A. salmonicida agar diffusion assay using PR-D9. Numbers represent the 1:2 dilution series, where dilution 1 is neat, dilution 2 is 1:2 dilution, dilution 3 is 1:4 dilution, etc.

FIG. 6A is a boxplot displaying Delta OD₆₀₀ readings from liquid broth assay for each compound concentration (mg/mL) with F. psychrophilum. The dashed line indicates no change in OD₆₀₀ over the incubation period. Groups with a * symbol are significantly different from the positive control using Dunnett's Test (p<0.05). “LBF”=lyophilized bacterial fermentation.

FIG. 6B is a photo of plating of 100 μL from combined wells of F. psychrophilum treated with PR-D9 (12.5 mg/mL).

FIG. 6C is a plate image displaying an F. psychrophilum agar diffusion assay using PR-D9. Numbers represent the 1:2 dilution series, where dilution 1 is neat, dilution 2 is 1:2 dilution, dilution 3 is 1:4 dilution, etc.

FIG. 7A is a boxplot displaying Delta OD₆₀₀ readings from liquid broth assay for each compound concentration (mg/mL) with P. damselae subsp. piscida. The dashed line indicates no change in OD₆₀₀ over the incubation period. Groups with a * symbol are significantly different from the positive control using Dunnett's Test (p<0.05). “LBF”=lyophilized bacterial fermentation.

FIG. 7B is a photo of plating of 100 μL P. damselae subsp. piscida from exposed to 12.5 mg/mL PR-D9 for 24 hours, incubated at 25° C. and checked at 48 hrs post-incubation.

FIG. 7C is a plate image displaying an P. damselae subsp. piscida agar diffusion assay using PR-D9. Numbers represent the 1:2 dilution series, where dilution 1 is neat, dilution 2 is 1:2 dilution, dilution 3 is 1:4 dilution, etc.

FIG. 8A is a boxplot displaying Delta OD₆₀₀ readings from liquid broth assay for each compound concentration (mg/mL) with V. anguillarum. The dashed line indicates no change in OD₆₀₀ over the incubation period. Groups with a * symbol are significantly different from the positive control using Dunnett's Test (p<0.05). “LBF”=lyophilized bacterial fermentation.

FIG. 8B is a photo of plating of 100 μL V. anguillarum from exposed to 12.5 mg/mL PR-D9 for 24 hours, incubated at 20° C. and checked at 48 hrs post-incubation.

FIG. 9A is a boxplot displaying Delta OD₆₀₀ readings from liquid broth assay for each compound concentration (mg/mL) with Y. ruckeri. The dashed line indicates no change in OD₆₀₀ over the incubation period. Groups with a * symbol are significantly different from the positive control using Dunnett's Test (p<0.05). “LBF”=lyophilized bacterial fermentation.

FIG. 9B is a photo of plating of 100 μL from combined wells of Y. ruckeri treated with 12.5 mg/mL PR-D9 and incubated for 72 hrs.

FIG. 9C is a plate image displaying an Y. ruckeri agar diffusion assay using PR-D9. Numbers represent the 1:2 dilution series, where dilution 1 is neat, dilution 2 is 1:2 dilution, dilution 3 is 1:4 dilution, etc.

FIG. 10A is a boxplot displaying Delta OD₆₀₀ readings from liquid broth assay for each compound concentration (mg/mL) with S. parasitica. The dashed line indicates no change in OD₆₀₀ over the incubation period. Groups with a * symbol are significantly different from the positive control using Dunnett's Test (p<0.05). “LBF”=lyophilized bacterial fermentation.

FIG. 10B is a plate image displaying an S. parasitica agar diffusion assay using PR-D9. Numbers represent the 1:2 dilution series, where dilution 1 is neat, dilution 2 is 1:2 dilution, dilution 3 is 1:4 dilution, etc.

FIG. 11A is a boxplot displaying Delta OD₆₀₀ readings from liquid broth assay for each compound concentration (mg/mL) with T. maritimum. The dashed line indicates no change in OD₆₀₀ over the incubation period. Groups with a * symbol are significantly different from the positive control using Dunnett's Test (p<0.05). “LBF”=lyophilized bacterial fermentation.

FIG. 11B is a plate image displaying a T. maritimum agar diffusion assay using PR-D9. Numbers represent the 1:2 dilution series, where dilution 1 is neat, dilution 2 is 1:2 dilution, dilution 3 is 1:4 dilution, etc.

FIG. 12A is a boxplot displaying Delta OD₆₀₀ readings from liquid broth assay for each compound concentration (mg/mL) with M. viscosa. The dashed line indicates no change in OD₆₀₀ over the incubation period. Groups with a * symbol are significantly different from the positive control using Dunnett's Test (p<0.05). “LBF”=lyophilized bacterial fermentation.

FIG. 12B is a plate image displaying an M. viscosa agar diffusion assay using PR-D9. Numbers represent the 1:2 dilution series, where dilution 1 is neat, dilution 2 is 1:2 dilution, dilution 3 is 1:4 dilution, etc.

FIG. 13A is a boxplot displaying Delta OD₆₀₀ readings from liquid broth assay for each compound concentration (mg/mL) with F. columnare. The dashed line indicates no change in OD₆₀₀ over the incubation period. Groups with a * symbol are significantly different from the positive control using Dunnett's Test (p<0.05). “LBF”=lyophilized bacterial fermentation.

FIG. 13B is a photo of plating of 100 μL from combined wells of F. columnare treated with 12.5 mg/mL PR-D9.

FIG. 13C is a plate image displaying an F. columnare agar diffusion assay using PR-D9. Numbers represent the 1:2 dilution series, where dilution 1 is neat, dilution 2 is 1:2 dilution, dilution 3 is 1:4 dilution, etc.

FIG. 14A is a boxplot displaying Delta OD₆₀₀ readings from liquid broth assay for each compound concentration (mg/mL) with R. salmoninarum. The dashed line indicates no change in OD₆₀₀ over the incubation period. Groups with a * symbol are significantly different from the positive control using Dunnett's Test (p<0.05). “LBF”=lyophilized bacterial fermentation.

FIG. 14B is a photo of plating of 100 μL from combined wells of R. salmoninarum treated with 12.5 mg/mL PR-D9.

FIG. 15 is a boxplot displaying Mean CT values for each compound concentration (mg/mL) with P. salmonis. Increasing CT values indicate diminished quantities of viable P. salmonis. Groups with a * symbol are significantly different from the positive control using Dunnett's Test (p<0.05).

FIG. 16A is a boxplot displaying relative quantities (RQ values) generated by a WSSV diagnostic TaqMan qPCR assay. The boxplot displays four biological replicates from PR-D9 dilutions. The positive control (Pos Ctrl) comprises 10 μL of WSSV homogenate and 190 μL PBS.

FIG. 16B is a boxplot displaying Mean CT values for each compound concentration (mg/mL) with White Spot Syndrome Virus. Increasing CT values indicate diminished quantities of viable WSSV particles. Groups with a * symbol are significantly different from the positive control using Dunnett's Test (p<0.05).

FIG. 17A is a chart showing the initial mean weight of the shrimp at the start of the study and end mean weight of the shrimp post 22 days of feeding. Mean weights were derived from tank bulk weight divided by number of shrimp in tanks.

FIG. 17B is a chart showing the mean weight gain of shrimp after feeding treatment diets for the duration of 22 days. The line in each treatment represents mean weight gain/shrimp; different shape dots in each treatment refer to mean weight gain/shrimp of replicate tanks. Treatments sharing letters are not statistically significant.

FIG. 17C is a mortality curve of shrimp fed with different treatment feeds post challenge (V. parahaemolyticus) over the duration of 10 days. Letters at the end of mortality curves show statistical significance. Mortality curves sharing letters are not statistically significant.

FIGS. 18A-C are plate images displaying a dilution series of 50% methanol extracts from two PR-D9 fermentations, FIG. 18A from one batch and FIG. 18B-C from a second batch, at time points of 14 hrs (FIG. 18B) and 22.5 hrs (FIG. 18A, 18C) applied to a Mueller Hinton+2% NaCl agar plate overlaid with a 0.005 OD culture of V. parahaemolyticus. Spots of inhibition are shown against V. parahaemiolyticus.

FIG. 19 is a plate image displaying whole PR-D9 cell broth with an excipient spotted onto a Mueller Hinton Agar+3% NaCl plate overlaid with V. parahaemolyticus. Excipients tested were OPS (upper left), sucrose (upper right), trehalose (lower left) and mannitol (lower right).

FIG. 20A is a plate image displaying an untreated (left) or a heat treated (right, 70° C. for 10 minutes) dilution series of PR-D9 whole fermentation broth spotted onto a Mueller Hinton+3% NaCl plate overlaid with V. parahaemolyticus.

FIG. 20B is a plate image displaying spot treatment of PR-D9 cells resuspended in PBS (left) or supernatant (right) after exposure to a range of temperature conditions. Temperature conditions from top to bottom were: 72° C. for 15 seconds, 90° C. for 5 minutes, 63° C. for 30 minutes, and an unheated control.

FIG. 20C is a plate image displaying a dilution series of PR-D9 lyophilized powder resuspended in PBS spotted onto a Mueller Hinton+3% NaCl agar plate overlaid with V. parahaemolyticus. Untreated control samples were resuspended in PBS and left at room temperature. The two autoclaved treatments were autoclaved in either water or 1×TAE for 15 minutes at 121° C. and 15 PSI.

FIGS. 21A and 21B are plate images displaying a 1:2 dilution series of spray dried PR-D9 (170° C. inlet and 90° C. outlet) resuspended at 25 mg/mL in 50% methanol and spotted onto a Mueller Hinton agar plate overlaid with a culture of V. parahaemolyticus.

FIG. 22 is a plate image displaying 3 microbial fermentation treatments applied to a soy-flower mannitol agar plate. Treatments are: (A) PR-D9, (B) Biomin Aquastar, and (C) INVE Pro-2.

### DETAILED DESCRIPTION

It is to be appreciated that certain aspects, modes, embodiments, variations and features of the present technology are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this present technology belongs.

### I. Definitions

The following terms are used herein, the definitions of which are provided for guidance.

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, “administration” of an agent, drug, bacterial strain or spore thereof, or composition of the present technology to a subject includes any route of introducing or delivering to a subject a compound to perform its intended function. Administration can be carried out by any suitable route, including orally, intranasally, parenterally (intravenously, intramuscularly, intraperitoneally, or subcutaneously), topically, or by inhalation. In some embodiments, the compositions of the present technology are formulated for enteric administration. In some embodiments, the compositions are formulated for oral, sublingual, or rectal delivery. In some embodiments, the compositions are formulated for use as a probiotic. In some embodiments, the compositions are formulated for use as a live biotherapeutic. As used herein, administration includes self-administration and administration by another.

As used herein, the terms “effective amount,” or “therapeutically effective amount,” and “pharmaceutically effective amount” refer to a quantity sufficient to achieve a desired therapeutic and/or prophylactic effect, e.g., an amount which results in the prevention of a disease, condition, and/or symptom(s) thereof. In the context of therapeutic or prophylactic applications, the amount of a composition administered to the subject will depend on the type and severity of the disease and on the characteristics of the subject, such as general health, age, sex, body weight, and tolerance to the composition drugs. It will also depend on the degree, severity, and type of disease or condition. The skilled artisan will be able to determine appropriate dosages depending on these and other factors. In some embodiments, multiple doses are administered. Additionally or alternatively, in some embodiments, multiple therapeutic compositions or compounds (e.g., pharmaceutical compositions comprising PD-R9 alone or in combination with additional therapeutics indicated for the target pathogen) are administered. In the methods described herein, compositions comprising the bacterial strain of the present technology, or spores thereof, may be administered to a subject having one or more signs, symptoms, or risk factors Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum, or combinations thereof. For example, a “therapeutically effective amount” of the compositions of the present technology, includes levels at which the presence, frequency, or severity of one or more signs, symptoms, or risk factors of Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum, or combinations thereof are, at a minimum, ameliorated. In some embodiments, a therapeutically effective amount reduces or ameliorates the physiological effects of, or the likelihood of developing Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum, or combinations thereof. In some embodiments, a therapeutically effective amount is achieved by multiple administrations. In some embodiments, a therapeutically effective amount is achieved with a single administration.

As used herein, “excipient” refers to substances and compositions that do not produce an adverse, allergic, or other untoward reaction when administered to an animal or a human and serve to increase the stability of the therapeutic agent during processing such as spray-drying, lyophilization, or some other treatment. As used herein, the term includes all inert, non-toxic, liquid or solid fillers, or diluents that do not react with the therapeutic substance of the invention in an inappropriate negative manner, solvents, dispersion media, coatings, antibacterial and antifungal agents, isotonic and absorption delaying agents, preservatives and the like, for example liquid pharmaceutical carriers e.g., sterile water, saline, sugar solutions, Tris buffer, ethanol, OPS, sucrose, trehalose, mannitol, glycerol, glycine, skim milk, bovine serum albumin (BS), lyophilization buffer, and/or certain oils.

As used herein, “fermentation” or “fermentation broth” or “whole fermentation” or “whole fermentation broth” refer to a liquid media growth of the referenced organism including media, whole organisms, and any extracellular factors.

As used herein, the terms “freeze-dried” or “freeze-drying” and “lyophilized” or “lyophilization” are used interchangeably and refer to a process that removes water from a product after it is frozen and placed under a vacuum and the products produced therefrom.

As used herein, the term “heat-stable” refers to a product, such as the bacterial strain of the present technology, that retains comparable activity after exposure to heat. In some embodiments, heat treatment comprises autoclaving. In some embodiments, the heat treatment may additionally or alternatively include passing the bacterial strain of the present technology through a feed mill pellet extruder. In some embodiments, the heat treatment may additionally or alternatively include passing the bacterial strain of the present technology through an industrial spray-dryer.

As used herein, “inhibit” or “inhibited” or “inhibition” of an organism refers to the stoppage of growth of said organism due to treatment with the strain of the present invention. In some embodiments, the inhibition can be bacteriostatic or bactericidal.

As used herein, “neutralize” or “neutralized” or “neutralization” of an organism refers to the permanent stoppage of growth of said organism due to treatment with the strain of the present invention.

As used herein, “PR-D9” or “D9” refers to the bacterial strain of the present technology having been deposited with the Agricultural Research Culture Collection (NRRL) International Depositary Authority on 28 Apr. 2021, under NRRL Accession Number B-68028, or variant strain thereof, or spore thereof, or compositions comprising the strain. The PR-D9 strain of the present technology is a strain of Paenibacillus elgii. In some embodiments, the PR-D9 strain of the present technology is a lyophilized bacterial fermentation and includes the bioactive components of the fermentation. In some embodiments, products comprising the PR-D9 bacterial strain of the present technology comprise live, viable cells. Additionally or alternatively, in some embodiments, products comprising the PR-D9 bacterial strain of the present technology are heat-killed and include actives of the PR-D9 bacterial strain in a pelleted feed or food additive. In some embodiments, the PR-D9 strain may be spray dried or lyophilized. In some embodiments, PR-D9 is formulated as a food or feed additive, such as an aquaculture food or an aquaculture feed additive. In some embodiments, PR-D9 is formulated as an aquaculture bath. In some embodiments, PR-D9 is formulated as a coating that can be sprayed onto or applied in various ways to feed pellets after pellet extrusion.

As used herein, “prevention,” “prevent,” or “preventing” of a disorder or condition refers to, in a statistical sample, reduction in the occurrence or recurrence of the disorder or condition in treated subjects/samples relative to an untreated controls, or refers delays the onset of one or more symptoms of the disorder or condition relative to the untreated controls.

As used herein, the terms “spray dried” or “spray drying” refer to a process that removes water from a product in a solution, suspension, dispersion, or emulsion by spraying the solution, suspension, dispersion, or emulsion into droplets which are subsequently dried by hot air and the products produced therefrom.

As used herein “subject” and “patient” are used interchangeably. In some embodiments, the subject is an animal subject. In some embodiments, the animal subject is a mammal. In some embodiments, the mammalian subject is a human. In some embodiments, the animal subject is a fish or crustacean. In some embodiments, the fish or crustacean is in an aquaculture environment.

“Treating,” “treat,” “treated,” or “treatment” of a disease or disorder includes: (i) inhibiting the disease or disorder, i.e., arresting its development; (ii) relieving the disease or disorder, i.e., causing its regression; (iii) slowing progression of the disorder; and/or (iv) inhibiting, relieving, or slowing progression of one or more symptoms of the disease or disorder.

As used herein, “variant” refers to a strain derived from a strain of the present technology by any means, such as, but not limited to, genetic engineering, radiation and/or chemical treatment, and/or selection, adaptation, screening, etc. In some embodiments, the variant is naturally occurring. In some embodiments, the variant is selected for or engineered. In some embodiments, the variant is a functionally equivalent variant, e.g., a variant that has the same, or improved, properties with respect to the inhibition of aquaculture pathogen growth as the mother strain. Such variants are a part of the present technology. In some embodiments, the term “variant” refers to a strain obtained by subjecting a strain of the present technology to any conventionally used mutagenization treatment including treatment with a chemical mutagen such as ethane methane sulphonate (EMPS) or N-methyl-N′-nitro-N-nitroguanidine (NTG), UV light, or to a spontaneously occurring variant. In embodiments in which variants are subjected to mutagenization, one of skill in the art may refer to the variant as a mutant. A variant may have been subjected to several mutagenization treatments (a single treatment should be understood as one mutagenization step followed by a screening/selection step). In some embodiments, less than 1%, less than 0.1%, less than 0.01%, less than 0.001%, or less than 0.0001% of nucleotides in the bacterial genome have been changed (such as by replacement, insertion, deletion, or a combination thereof) compared to the mother strain. Such variants, which may be identified by using appropriate screening techniques, are a part of the present technology.

It is to be appreciated that the various modes of treatment or prevention of medical diseases and conditions as described are intended to mean “substantial,” which includes total but also less than total treatment or prevention, and wherein some biologically or medically relevant result is achieved.

### II. Vibrio parahaemolyticus Infection

Vibrio parahaemolyticus is a gram-negative, rod-shaped, and is facultatively aerobic bacterium. The bacterium causes gastroenteritis in immunocompromised humans and is a significant pathogen of shrimp aquaculture, causing Acute Hepatopancreatic Necrosis Disease (AHPND) or Early Mortality Syndrome (EMS). AHPND is typically manifested by pale atrophied hepatopancreas with empty stomach and midgut, causing losses in post larvae and juveniles attributing to global losses of >$1 billion/year. The pathogen reaches host by means of oral ingestion of contaminated materials e.g. cannibalism of infected individual, and colonizes digestive system damaging the hepatopancreas. Sustainable prevention and control measures against the disease are actively sought by the industry since resistance to multiple antibiotics have been recently reported in the pathogen.

In some embodiments, the present technology provides methods and compositions for the treating or preventing V. parahaemolyticus infection, including reducing the severity of one or more risk factors, signs, or symptoms associated with V. parahaemolyticus infections. In some embodiments, the compositions comprise the novel Paenibacillus strain PR-D9, or variant strain thereof, or spores thereof.

### III. White Spot Syndrome Virus Infection

White Spot Syndrome Virus is a large DNA virus that represents a persistent problem for shrimp aquaculture and treatment options are currently in demand. Vertical transmission occurs from infected broodstock and horizontal transmission often occurs via cannibalism of sick or dying prawns or directly through contaminated water. Infection leads to the rapid onset of mass mortality, frequently 80% or more of a population, with symptoms including lethargy, cessation of feedings, moribund behavior, loose carapace, high degrees of color variation, white midgut line, white calcium deposits in shell, and a delayed clotting reaction.

In some embodiments, the present technology provides methods and compositions for the treating or preventing White Spot Syndrome Virus infection, including reducing the severity of one or more risk factors, signs, or symptoms associated with White Spot Syndrome Virus infections. In some embodiments, the compositions comprise the novel Paenibacillus strain PR-D9, or variant strain thereof, or spores thereof.

### IV. Aeromonas salmonicida Infection

A. salmonicida is a gram-negative, rod-shaped, facultative anaerobe infecting a wide range of salmonids and is the etiological agent of furunculosis. Symptoms in infected fish include internal hemorrhaging, swelling of vents and kidneys, boils, ulcers, liquefaction, and gastroenteritis. A. salmonicida is found ubiquitously at fish farms and increasing anti-bacterial resistance poses a major problem to the industry.

In some embodiments, the present technology provides methods and compositions for the treating or preventing A. salmonicida infection, including reducing the severity of one or more risk factors, signs, or symptoms associated with A. salmonicida infections. In some embodiments, the compositions comprise the novel Paenibacillus strain PR-D9, or variant strain thereof, or spores thereof.

### V. Flavobacterium psychrophilium Infection

F. psychrophilium is etiological agent of bacterial cold water disease (BCWD), with symptoms including tissue erosion, jaw ulcerations, inflammation, and fin pathologies. This bacterium is gram-negative and rod-shaped, infecting a broad range of hosts inhabiting freshwater, usually at temperatures <13° C. The bacterium is especially lethal to rainbow trout fry and remains a significant challenge for that industry.

In some embodiments, the present technology provides methods and compositions for the treating or preventing F. psychrophilium infection, including reducing the severity of one or more risk factors, signs, or symptoms associated with F. psychrophilium infections. In some embodiments, the compositions comprise the novel Paenibacillus strain PR-D9, or variant strain thereof, or spores thereof.

### VI. Photobacterium damselae piscida Infection

P. damselae piscida is gram-negative, rod-shaped, and is not mobile. The bacterium has a wide host range in the marine environment and causes photobacteriosis, characterized by granulatomous lesions and necrosis of several visceral organs. The bacteria has a significant, detrimental economic impact in the yellowtail, seabream, and sea bass farming industries.

In some embodiments, the present technology provides methods and compositions for the treating or preventing P. damselae piscida infection, including reducing the severity of one or more risk factors, signs, or symptoms associated with P. damselae piscida infections. In some embodiments, the compositions comprise the novel Paenibacillus strain PR-D9, or variant strain thereof, or spores thereof.

### VII. Vibrio anguillarum Infection

V. anguillarum is a gram-negative, arcuate-rod bacteria with one polar flagellum and infects a wide range of aquatic species (e.g., fish bivalves, mollusks, and crustaceans) causing vibriosis. Vibriosis is characterized by a haemorrhagic septicaemia, presenting with darkened fins, ulcers, and sepsis. Controlling disease in aquaculture is a challenge to the aquatic farming industry due to concerns over the spread of antibacterial resistance.

In some embodiments, the present technology provides methods and compositions for the treating or preventing V. anguillarum infection, including reducing the severity of one or more risk factors, signs, or symptoms associated with V. anguillarum infections. In some embodiments, the compositions comprise the novel Paenibacillus strain PR-D9, or variant strain thereof, or spores thereof.

### VIII. Yersinia ruckeri Infection

Y. ruckeri is a gram-negative, rod-shaped, and facultative anaerobe bacterium which infects a wide range of salmonids causing enteric redmouth (ERM) disease. ERM presents with low initial mortality rates that climb rapidly when fish are exposed to stressors in the farm ecosystem. Symptoms can include dark coloration, loss of appetite, lethargy, and reddening due to subcutaneous hemorrhages. Y. ruckeri outbreaks have a serious economic impact worldwide on fish farms and vaccination is inconsistently protective.

In some embodiments, the present technology provides methods and compositions for the treating or preventing Y. ruckeri infection, including reducing the severity of one or more risk factors, signs, or symptoms associated with Y. ruckeri infections. In some embodiments, the compositions comprise the novel Paenibacillus strain PR-D9, or variant strain thereof, or spores thereof.

### IX. Streptococcus agalactiae Infection

S. agalactiae is a gram-positive, facultative anaerobe infecting a wide range of mammalian, reptile, amphibian, and fish hosts. Notably, S. agalactiae infects Nile tilapia (Oreochromisniloticus) causing meningitis and high rates of mortality during outbreaks on farms. Symptoms of infection in fish can include hemorrhages, cornea opacity, spinning near water surface, erosion of the caudal fin, and eye protrusion.

In some embodiments, the present technology provides methods and compositions for the treating or preventing S. agalactiae infection, including reducing the severity of one or more risk factors, signs, or symptoms associated with S. agalactiae infections. In some embodiments, the compositions comprise the novel Paenibacillus strain PR-D9, or variant strain thereof, or spores thereof.

### X. Streptococcus iniae Infection

S. iniae is a gram-positive bacterium that infects fish and human hosts. Notably, S. iniae infects Nile tilapia and rainbow trout causing meningitis and high rates of mortality during outbreaks. Symptoms of infection in can include lethargy, dorsal rigidity, erratic swimming behavior, septicemia, and damage to the central nervous system.

In some embodiments, the present technology provides methods and compositions for the treating or preventing S. iniae infection, including reducing the severity of one or more risk factors, signs, or symptoms associated with S. iniae infections. In some embodiments, the compositions comprise the novel Paenibacillus strain PR-D9, or variant strain thereof, or spores thereof.

### XI. Piscrickettsia salmonis Infection

P. salmonis is a gram-negative bacterium that causes septicemia in salmonids, resulting in anemia, kidney necrosis, enlarged spleen, hemorrhages, liver nodules, dark coloration, skin lesions, lack of appetite, anorexia, and lethargic swimming. The disease, known as piscirickettiosis, most often occurs in salt water, but transmission and disease can occur in freshwater. Outbreaks of disease are a major driver of antibiotic use, especially in Chile, and thus novel therapies are highly sought after.

In some embodiments, the present technology provides methods and compositions for the treating or preventing P. salmonis infection, including reducing the severity of one or more risk factors, signs, or symptoms associated with P. salmonis infections. In some embodiments, the compositions comprise the novel Paenibacillus strain PR-D9, or variant strain thereof, or spores thereof.

### XII. Saprolegnia parasitica Infection

S. parasitica is a water mold (oomycete) that represents a persistent problem in freshwater aquaculture. The oomycete causes saprolegniosis, with symptoms including visible white or grey patches of mycelium on the fish and moribund behavior prior to death. Several husbandry practices and treatments are used for control with varying success.

In some embodiments, the present technology provides methods and compositions for the treating or preventing S. parasitica infection, including reducing the severity of one or more risk factors, signs, or symptoms associated with S. parasitica infections. In some embodiments, the compositions comprise the novel Paenibacillus strain PR-D9, a variant strain thereof, or spores thereof.

### XIII. Tenacibaculum maritimum Infection

T. maritimum is a gram-negative, motile, rod-shaped bacterium infecting numerous marine species of fish (e.g., Atlantic salmon, turbot, sea bream) and is one of the etiological agents of tenacibaculosis. Tenacibaculosis is an ulcerative disease with symptoms including ulcers, necrosis, eroded mouth, frayed fins and gills, and necrosis of the gills and eyes. Vaccinations against T. maritimum exist but can require antibiotic supplementation.

In some embodiments, the present technology provides methods and compositions for the treating or preventing T. maritimum infection, including reducing the severity of one or more risk factors, signs, or symptoms associated with T. maritimum infections. In some embodiments, the compositions comprise the novel Paenibacillus strain PR-D9, a variant strain thereof, or spores thereof.

### XIV. Moritella viscosa Infection

M. viscosa is a gram-negative anaerobe, primarily infecting salmonids in seawater <10° C. and is the etiological agent of winter ulcer disease. Disease symptoms include ulcers, pale gills and fin rot.

In some embodiments, the present technology provides methods and compositions for the treating or preventing M. viscosa infection, including reducing the severity of one or more risk factors, signs, or symptoms associated with M. viscosa infections. In some embodiments, the compositions comprise the novel Paenibacillus strain PR-D9, a variant strain thereof, or spores thereof.

### XV. Flavobacterium columnare Infection

F. columnare is a gram-negative, motile, and rod-shaped bacteria and infects a wide range of freshwater fish species (e.g., catfish, tilapia, carp) causing columnaris disease. Symptoms of columnaris disease include skin lesions, fin erosion, and gill necrosis with a high degree of mortality and subsequent economic loss.

In some embodiments, the present technology provides methods and compositions for the treating or preventing F. columnare infection, including reducing the severity of one or more risk factors, signs, or symptoms associated with F. columnare infections. In some embodiments, the compositions comprise the novel Paenibacillus strain PR-D9, a variant strain thereof, or spores thereof.

### XVI. Renibacterium salmoninarum Infection

R. salmoniarum is a gram-positive, intracellular bacterium infecting salmonids and causing bacterial kidney disease (BKD). Symptoms of BKD include exophthalmia, ulcerations, hemorrhages, and swelling and hemorrhages of internal organs such as the kidney, heart, spleen, and liver. The bacterium can be vertically and horizontally transmitted and is therefore a major concern for both broodstock and juvenile fish. Antibiotic treatments are available but prolonged treatment is required and progress can be slow.

In some embodiments, the present technology provides methods and compositions for the treating or preventing R. salmoniarum infection, including reducing the severity of one or more risk factors, signs, or symptoms associated with R. salmoniarum infections. In some embodiments, the compositions comprise the novel Paenibacillus strain PR-D9, a variant strain thereof, or spores thereof.

### XVII. Therapeutic and Prophylactic Methods

The following discussion is presented by way of example only, and is not intended to be limiting.

One aspect of the present technology includes methods of reducing the risk of, preventing, or treating infection with any one of Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum, or combinations thereof, in a subject diagnosed as having, suspected as having, or at risk of having an infection associated with any one or more of the pathogens. In therapeutic applications, compositions or medicaments comprising a PR-D9 bacterial strain, variant strain thereof, or spore thereof, are administered to a subject suspected of, or already suffering from such a disease (such as, e.g., the symptoms associated with the pathogenic infections described herein), in an amount sufficient to cure, or at least partially arrest, the symptoms of the disease, including its complications and intermediate pathological phenotypes in development of the disease.

Subjects suffering from infection by Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum or combinations thereof can be identified by any or a combination of diagnostic or prognostic assays known in the art.

In some embodiments, subjects infected by Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum or combinations thereof treated with the bacterial strains of the present technology, or spores thereof, will show amelioration or elimination of one or more of the symptoms associated with the pathogenic infections described herein.

In one aspect, the present technology provides a method for preventing or delaying the onset, or development of symptoms, of Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum, or combinations thereof in a subject at risk for any of these pathogens. In some embodiments, the bacterial strain of the present technology, or a variant strain thereof, is formulated as a probiotic useful as a food supplement and for re-establishing beneficial bacteria in the intestinal tract. In some embodiments, the bacterial strain of the present technology, or a variant strain thereof, is formulated as a live biotherapeutic product useful in pharmaceutical applications.

One aspect of the present technology includes methods of treating or preventing infection by Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum, or combinations thereof, in a subject diagnosed as having, suspected as having, or at risk of having infection by any of the pathogens. In therapeutic applications, compositions or medicaments comprising a bacterial strain, or a variant strain thereof, or spore thereof, of PR-D9 are administered to a subject suspected of, or already suffering from such a disease, in an amount sufficient to cure, or at least partially arrest, the symptoms of the disease, including its complications and intermediate pathological phenotypes in development of the disease.

Subjects suffering from infection by Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum, or combinations thereof, can be identified by any or a combination of diagnostic or prognostic assays known in the art.

In some embodiments, infection by Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum, or combinations thereof, is treated with the bacterial strain of the present technology, or a variant strain thereof, or spores thereof, in subjects showing amelioration or elimination of one or more of the symptoms associated with the pathogenic infections described herein.

In one aspect, the present technology provides a method for preventing or delaying the onset of infection by or symptoms of infection by Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum, or combinations thereof, in a subject at risk of having such an infection. In some embodiments, the bacterial strain of the present technology, or a variant strain thereof, is formulated as a probiotic useful as a food supplement and for reestablishing beneficial bacteria in the intestinal tract. In some embodiments, the bacterial strain, or a variant strain thereof, of the present technology is formulated as a live biotherapeutic product useful in pharmaceutical applications. In some embodiments, the bacterial strain of the present technology, or a variant strain thereof, is formulated as a food or feed additive. In some embodiments, the food or feed additive is an aquaculture food or aquaculture feed additive. In some embodiments, the bacterial strain of the present technology, or a variant strain thereof, is formulated as an aquaculture bath.

### XVIII. Modes of Administration and Effective Dosages

Compositions of the present technology for use in preventing, ameliorating, or treating infections with Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum, or combinations thereof, and/or reducing the severity of one or more risk factors, signs, or symptoms associated with infection include live probiotic PR-D9 Paenibacillus bacteria, or a variant strain thereof, according to the present technology, provided in the form of vegetative cells and/or spores. In addition to the vegetative cells and or spores, the supernatant of fermentation containing PR-D9 actives is concentrated via lyophilization or spray-drying, such that the final product is a concentrated whole fermentation broth with cytoprotectants or excipients added per application. In some embodiments, the bacterial strain, or a variant strain thereof, is a lyophilized bacterial fermentation. In some embodiments, the bacterial strain, or a variant strain thereof, is lyophilized. In some embodiments the bacterial strain, or a variant strain thereof, is spray dried. In some embodiments, the bacterial strain, or a variant strain thereof, is heat treated. In some embodiments, the bacterial strain, or a variant strain thereof, is autoclaved. The compositions of the present technology are administered to the subject in effective amounts (e.g., amounts that have desired therapeutic effect). The dose and dosage regimen will depend upon the degree of the infection in the subject, the characteristics of the Paenibacillus strain used, e.g., its therapeutic index, the subject, and the subject's history. The effective amount may be determined during pre-clinical trials and clinical trials by methods familiar to veterinarians, aquaculture farmers, physicians, and/or clinicians.

Compositions of the present technology may be formulated for adding to food, or used directly as a food supplement. In some embodiments, the food or food supplement is an aquaculture food or aquaculture food supplement. The formulation may further include other probiotic agents or nutrients for promoting spore germination and/or bacterial growth.

Additional components of the compositions of the present technology may include an excipient. In some embodiments, the excipient comprises one or more lyoprotectants, including, but not limited to, mannitol, trehalose, sucrose, OPS, glycerol, glycine, skim milk, bovine serum albumin (BSA), lyophilization buffer, and/or any other known protectant.

In some embodiments, the compositions of the present technology, such as the PR-D9 lyophilized bacterial fermentation product, are added to a bulk carrier such as, but not limited to, rice hulls, wheat hulls, calcium carbonate (limestone), whey, maltodextrin, sucrose, dextrose, yeast culture, dry starch, and/or sodium aluminosilicate.

In some embodiments, the compositions of the present technology may include a preservative selected from the group consisting of sucrose, sodium ascorbate, and glutathione. In some embodiments the preservative is a cryoprotectant selected from the group consisting of a nucleotide, a disaccharide, a polyol, and a polysaccharide. In some embodiments, the cryoprotectant is selected from the group consisting of inosine-5′-monophosphate (IMP), guanosine-5′-monophosphate (GMP), adenosine-5′-monophosphate (AMP), uranosine-5′-monophosphate (UMP), cytidine-5′-monophosphate (CMP), adenine, guanine, uracil, cytosine, guanosine, uridine, cytidine, hypoxanthine, xanthine, orotidine, thymidine, inosine, trehalose, maltose, lactose, sucrose, sorbitol, mannitol, dextrin, inulin, sodium ascorbate, glutathione, skim milk, and cryoprotectant 18.

The Paenibacillus bacterial strain described herein, or a variant strain thereof, can be incorporated into pharmaceutical compositions for administration, singly or in combination with other agents or bacterial strains, and given to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions. Carriers can be solid-based dry materials for formulations in powdered form, and can be liquid or gel-based materials for formulations in liquid or gel forms, which forms depend, in part, upon the routes or modes of administration. In some embodiments, the pharmaceutically acceptable carrier comprises a polysaccharide, locust bean gum, an anionic polysaccharide, a starch, a protein, sodium ascorbate, glutathione, trehalose, sucrose, or pectin. In some embodiments, the polysaccharide comprises a plant, animal, algal, or microbial polysaccharide. In some embodiments, the polysaccharide comprises guar gum, inulin, amylose, chitosan, chondroitin sulphate, an alginate, or dextran. In some embodiments, the starch comprises rice starch.

Pharmaceutical compositions are typically formulated to be compatible with its intended route of administration. Examples of routes of administration include enteric (e.g., oral, sublingual, rectal) administration. A therapeutic composition can be formulated to be suitable for oral administration in a variety of ways, for example in a liquid, a powdered food supplement, a solid food, a packaged food, a wafer, tablets, troches, or capsules, e.g., gelatin capsules, and the like. In some embodiments, the therapeutic compositions of the present technology comprise lyophilized, spray dried, heat-treated, autoclaved, or some combination thereof of PR-D9, or a variant strain thereof. In some embodiments, the lyophilized, spray dried, heat-treated, autoclaved, or some combination thereof of PR-D9, or a variant strain thereof, is encapsulated. A therapeutic composition can be formulated to be suitable for rectal administration in a variety of ways, for example in a suppository, liquid enema, or foam. Other formulations will be readily apparent to one skilled in the art. pH can be adjusted with acids or bases, such as hydrochloric acid or sodium hydroxide.

For aquaculture applications, the pharmaceutical compositions of the present technology may be formulated as a fish feed or fish feed additive or as a bath (immersion) treatment agent.

Dosage, toxicity and therapeutic efficacy of any therapeutic agent can be determined by standard pharmaceutical procedures in cell cultures or experimental animals. The data obtained from the cell culture assays and animal studies can be used in formulating a range of dosage for use in fish. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in various fish species accurately.

In some embodiments, the compositions of the present technology contain 0.025-12.5 mg/mL of lyophilized, spray dried, heat-treated, autoclaved, or some combination thereof of Paenibacillus (i.e., PR-D9) bacterium, or a variant strain thereof, or bacterial spore thereof. In some embodiments, the compositions of the present technology contain 0.78-0.025 mg/mL of lyophilized, spray dried, heat-treated, autoclaved, or some combination thereof of Paenibacillus (i.e., PR-D9) bacterium or bacterial spore. In some embodiments, when formulated as a feed additive, the compositions of the present technology contain inclusion levels of 0.1% or less to 20% or more (w/w) of feed of PR-D9 bacterium, or a variant thereof, or bacterial spore thereof. In some embodiments, the compositions, when formulated as a feed additive, contain about 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.30 7.4% 7.50 7.6%, 7.700 7.8%, 7.9%, 8.0%, 8.1%, 8.2%, 8.3%, 8.4%, 8.5%, 8.6%, 8.7%, 8.8%, 8.9%, 9.0%, 9.100, 9.2%, 9.3%, 9.4%, 9.5%, 9.6%, 9.7%, 9.8%, 9.9%, or 10% (w/w) of feed of PR-D9 bacterium, or a variant thereof, or bacterial spore thereof. In some embodiments, when formulated as a feed additive, the compositions of the present technology contain inclusion levels of about 0.25% (w/w) of feed of PR-D9 bacterium, or a variant thereof, or bacterial spore thereof. In some embodiments, when formulated as a feed additive, the compositions of the present technology contain inclusion levels of about 0.5% (w/w) of feed of PR-D9 bacterium, or a variant thereof, or bacterial spore thereof. In some embodiments, when formulated as a feed additive, the compositions of the present technology contain inclusion levels of about 1% (w/w) of feed of PR-D9 bacterium, or a variant thereof, or bacterial spore thereof. In some embodiments, when formulated as a feed additive, the compositions of the present technology contain inclusion levels of about 5% (w/w) of feed of PR-D9 bacterium, or a variant thereof, or bacterial spore thereof. In some embodiments, when formulated as a feed additive, the compositions of the present technology contain inclusion levels of about 10% (w/w) of feed of PR-D9 bacterium, or a variant thereof, or bacterial spore thereof. In some embodiments, when formulated as a feed additive, the compositions of the present technology contain inclusion levels of less than 5% (w/w) of feed of PR-D9 bacterium, or a variant thereof, or bacterial spore thereof. In some embodiments, when formulated as a feed additive, the compositions of the present technology contain inclusion levels of less than 1% (w/w) of feed of PR-D9 bacterium, or a variant thereof, or bacterial spore thereof. In some embodiments, the compositions of the present technology are delivered as lyophilized, spray dried, heat-treated, autoclaved, or some combination thereof of material or powder to be re-suspended for oral delivery or packaged into capsules or as a fish feed or as a fish feed additive or as a water bath treatment. The lyophilized, spray dried, heat-treated, autoclaved, or some combination thereof of material and capsules may be coated for better enteric stability.

An exemplary treatment regimen entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the subject can be administered a prophylactic regime. In some embodiments, compositions of the present technology are administered to a subject once, twice, or three times per day for 10 to 14 days or until the subject is deemed cured of primary disease, not to be at risk for recurrence of primary disease, or not to be at risk for contracting the disease. In some embodiments, administration is paired with a shortened exposure to agents known in the art for the treatment of the pathogenic infections described herein, followed by once, twice, or three times daily dosing for 10 to 14 days or until the patient is deemed cured of primary disease or not to be at risk for recurrence of the disease. In some embodiments, methods of prophylaxis comprise administration of compositions of the present technology once, twice, or three times daily for 10 to 14 days or until the patient is deemed not to be at risk of contracting the disease.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

### XIX. Combination Therapy with Paenibacillus Strain of the Present Technology

In some embodiments, the Paenibacillus strain of the present technology, or a variant strain thereof, or spores thereof, may be combined with one or more additional therapies for the prevention or treatment of the pathogenic infections described herein.

In some embodiments, an additional therapeutic agent is administered to a subject in combination with the Paenibacillus strain of the present technology, or a variant strain thereof, or spores thereof, such that a synergistic therapeutic effect is produced. For example, administration of PD-R9 with one or more additional therapeutic agents for the prevention or treatment of the pathogenic infections described herein will have greater than additive effects in the prevention or treatment of the disease.

In any case, the multiple therapeutic agents may be administered in any order or even simultaneously. If simultaneously, the multiple therapeutic agents may be provided in a single, unified form, or in multiple forms. One of the therapeutic agents may be given in multiple doses, or both may be given as multiple doses. In addition, the combination methods, compositions and formulations are not to be limited to the use of only two therapeutics.

### EXAMPLES

The following examples are provided by way of illustration only and not by way of limitation. Those of skill in the art will readily recognize a variety of non-critical parameters that could be changed or modified to yield essentially the same or similar results. The examples should in no way be construed as limiting the scope of the present technology, as defined by the appended claims. For each of the examples below, any pathogen described herein could be used and any means of treatment preparation described herein could be used. By way of example, the pathogens treated in the examples below could be Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum, or combinations thereof, treated with PR-D9 that has been spray dried, lyophilized, or otherwise treated.

PR-D9 preparation. PR-D9 was isolated according to Example 1 (described below). Stable isolates of the PR-D9 strain were made in the laboratory and stored. Briefly, PR-D9 stocks, stored in 20% glycerol at −80° C., were thawed and grown in 1 L seed flasks of liquid media and then transferred to a 6 L bio-reactor (Infors) for growth over 24-48 hours. The whole fermentation broth, including the cell mass, supernatant, spent media components, and any secondary metabolites, was then lyophilized to a powder, ground, and stored until use. Sometimes, an excipient, such as trehalose was added prior to lyophilization to increase the survival of the strains. The lyophilized powder was used as specified below, including being resuspended and grown in media or being added as a feed component.

### General Materials and Methods for Examples 2-16.

Bacterial preparation. Bacteria were streaked from cryopreserved stock onto appropriate media, incubated, and grown in broth according to Table 1. Preparation of S. parasitica was completed similarly to methods described for bacteria, except a loop from a single spore colony (SSC) of hyphae was selected and placed into broth. Media preparation followed guidelines stipulated in SOP/CATC/4105, while pathogen preparation followed SOP/CATC/4106. Bacterial OD's were recorded throughout growth time.

TABLE 1
Pathogens and growth conditions. Media types included tryptic soy agar and broth (TSA/TSB)
with salt (TSA2/TSB2) or 5% yeast (TSAY/TSBY), tryptone yeast extract salts agar (TYES),
charcoal agar (KDM2), marine agar and broth (MA/MB), and glucose yeast media (GY).

| | Temp. | | Plate | | Broth | | Shaking | Agar | | Broth |
|---|---|---|---|---|---|---|---|---|---|---|
| Pathogen | (° C.) | Media | Time | | Time | | (RPM) | Assay | | (mL)^{a} |
| V. parahaemolyticus | 30 | TSA2/TSB2 | 22 | hrs | 18 | hrs | 150 | 24 | hrs | 5 |
| S. agalactiae | 28 | TSAY/TSBY | 4 | days | 48 | hrs | 150 | 50 | hrs | 3 |
| S. iniae | 28 | TSAY/TSBY | 4 | days | 48 | hrs | 150 | 24 | hrs | 3 |
| M. viscosa | 15 | TSA2/TSB2 | 5 | days | 48 | hrs | None | 4 | days | 3 |
| F. psychrophilium | 15 | TYES | 7 | days | 5 | days | None | 7 | days | 3 |
| A. salmonicida | 20 | TSA/TSB | 5 | days | 22 | hrs | 200 | 24 | hrs | 3 |
| F. columnare | 25 | TYES | 4 | days | 72 | hrs | None | 74 | hrs | 10 |
| P. damselae piscida | 25 | TSA2/TSB2 | 48 | hrs | 18 | hrs | 150 | 7 | hrs | 10 |
| R. salmoninarum | 15 | KDM2 | 29 | days | 14 | days | 200 | 14 | days | 10 |
| S. parasitica | 20 | GY | 6 | days | 5 | days | None | N/Ap | | 45 |
| T. maritimum | 15 | MA/MB | 7 | days | 7 | days | None | 7 | days | 10 |
| V. anguillarum | 20 | TSA2/TSB2 | 48 | hrs | 22 | hrs | 200 | 48 | hrs | 3 |
| Y. ruckeri | 20 | TSA | 48 | hrs | 22 | hrs | None | 24 | hrs | 3 |

^{a}One colony selected for small broth culture.

PR-D9 preparation. PR-D9 was serially diluted from stock concentrations to provide 10 dilutions, each of 1:1 in PBS. Final concentrations for each dilution are provided in Table 2.

TABLE 2
Stock and diluted concentrations of PR-D9.
These concentrations are sued in the Agar Diffusion
Assay, while a 1 in 2 dilution with bacterial broth was
completed for group in the Liquid Broth Assay.

| Dilution Number | PR-D9 Concentration (mg/mL) |
|---|---|
| 1 | 25.00 |
| 2 | 12.50 |
| 3 | 6.25 |
| 4 | 3.13 |
| 5 | 1.56 |
| 6 | 0.78 |
| 7 | 0.39 |
| 8 | 0.20 |
| 9 | 0.10 |
| 10 | 0.05 |

Liquid broth assay. Two batches of PR-D9 were assayed (“Batch No. 1” and “Batch No. 2”). The first batch of PR-D9 was used for all pathogens except M. viscosa, T. maritumum, S. parasitica, and F. columnare, which were treated with the second batch of PR-D9. Optical density (OD₆₀₀) readings of bacterial media stock were recorded. 100 μL of PR-D9 as per Table 2 were added to each well. Then, 100 μL of bacteria was added to each well, covered, and OD₆₀₀ readings were captured for each well and the plate was incubated per Table 3. Control (100 μL PBS+100 μL pathogen), PR-D9 Control (100 μL neat PR-D9+100 μL media), and Blank Control (100 μL PBS+100 μL media) were also plated. After incubation, OD₆₀₀ readings were captured for each well and normalized to the Blank Control. All OD₆₀₀ readings were corrected based on initial OD₆₀₀ readings (i.e., just prior to beginning the assay) and were reported as delta OD₆₀₀ (final read subtract initial read).

TABLE 3
Incubation parameters for liquid broth assays.

| | | | | | PR-D9 |
|---|---|---|---|---|---|
| | Temp. | | Incubation | | Batch |
| Pathogen | (° C.) | Media | Time | CFU/mL | No. |
| A. salmonicida | 20 | TSA/TSB | 48 hrs | 3.6 × 10⁸ | 1 |
| F. psychrophilium | 15 | TYES | 7 days | 7.7 × 10⁷ | 1 |
| F. columnare | 25 | TYES | 3 days | 7.0 × 10⁶ | 2 |
| M. viscosa | 15 | TSA2/TSB2 | 4 days | 2.2 × 10⁸ | 2 |
| P. damselae | 25 | TSA2/TSB2 | 7 hrs | 2.7 × 10⁸ | 1 |
| piscida | | | | | |
| R. salmoninarum | 15 | KDM2 | 14 days | 3.0 × 10⁸ | 1 |
| S. agalactiae | 28 | TSAY/TSBY | 4 days | 4.7 × 10⁸ | 1 |
| S. iniae | 28 | TSAY/TSAB | 24 hrs | 5.8 × 10⁷ | 1 |
| S. parasitica | 20 | GY | 4 days | ND^{A} | 2 |
| T. maritimum | 15 | MA/MB | 7 days | 1.8 × 10⁸ | 2 |
| V. anguillarum | 20 | TSA2/TSB2 | 24 hrs | 1.4 × 10⁸ | 1 |
| V. | 30 | TSA2/TSB2 | 3 days | 4.7 × 10⁷ | 1 |
| parahaemolyticus | | | | | |
| Y. ruckeri | 20 | TSA | 3 days | 1.4 × 10⁸ | 1 |

Agar diffusion assay. For the agar diffusion assays, 45 μL of PR-D9 (neat) were removed and a 20 μL aliquot was placed into a new tube. The remaining 25 μL were diluted 1 in 2 using PBS to yield Concentration 2. The dilution step was then repreated upt to Concentration 10 (Table 4). All groups were plated in quadruplicate. 5 μL of each PR-D9 concertation was pipetted onto an agar plate containing a lawn of bacteria. Each dilution number was labelled on the bottom of the plate, which included a control (5 μL PBS) on each plate. Plates were incubated as per Table 3 and each concentration was monitored for zones of inhibition post-incubation. If inhibition was observed, the diameter was measured and recorded. Batch No. 2 of PD-R9 was used to treat M. viscosa, T. maritumum, S. parasitica, and F. columnare. Batch No. 1 was used for all other bacteria.

Alternative plate assays. Two pathogens, White Spot Syndrome Virus (WSSV) and P. salmonis, could not be analyzed by broth or agar growth inhibition assays due to lack of growth without cells. Instead, a plate assay whereby pathogens were incubated with PR-D9 for 24 hrs in media to maintain pathogen viability was used.

qPCR Assays. qPCR plate assays were used to measure white spot syndrome virus and P. salmonis viability after PR-D9 exposure. Both pathogens were incubated with a dilution series of PR-D9 for 24 hours, DNA was extracted, and qPCR was performed. A positive control (media with pathogen and PBS), a PR-D9 control (media without pathogen and with PR-D9), and a media blank (media and PBS only) were also assayed. All samples were analyzed in quadruplicate.

Data analysis. Liquid broth assays were read via spectrophotometry (OD₆₀₀) before and after incubation with PR-D9. Each PR-D9 concentration was run in quadruplicate and all wells were corrected against a blank sample containing 100 μL of each of PBS and media. Initial readings were then subtracted from final readings to produce delta OD₆₀₀. Delta OD₆₀₀ readings were compared with ANOVA and post-hoc Dunnett's Multiple Comparison Test (p<0.05), which compares each treatment group to the control.

Data from the agar diffusion assays were not analyzed statistically, but rather descriptively. The presence or absence of growth inhibition and the diameter of inhibition is reported for each dilution of PR-D9.

Alternative Plate assays were quantified using cycle threshold (CT) values produced from qPCR. The study design was similar to that of the liquid broth assay, but rather than analyzing delta OD₆₀₀, CT values of quadruplicate treated wells were compared to positive controls. All CT values were produced from post-incubated samples.

### Example 1: Isolation of Strains and Discovery of PR-D9 as a Strain Effective in Methods for Inhibiting Aquaculture Pathogen Growth

Strain isolation from soil samples. The isolation of microbes for use as direct fed microbials in aquaculture followed a standard method of isolation. Briefly, soil samples were collected from various locations and stored at room temperature in suitable containers (e.g., 50 mL Falcon tubes or plastic bags) until return to the laboratory where debris, such as rocks or woody material, was filtered out. Then, 1 g of soil was suspended in 10 mL of sterile water and serially diluted 1:10 out to 1E-6. 100 μL of each dilution was plated on various standard agar media, including TSA, LB, and actinomycetes isolation agar (AIA), or other standard microbiological media. At times, 2-3% NaCl was added to simulate marine agar conditions. Plates were incubated at 27-32° C. for 1-7 days until colonies appeared.

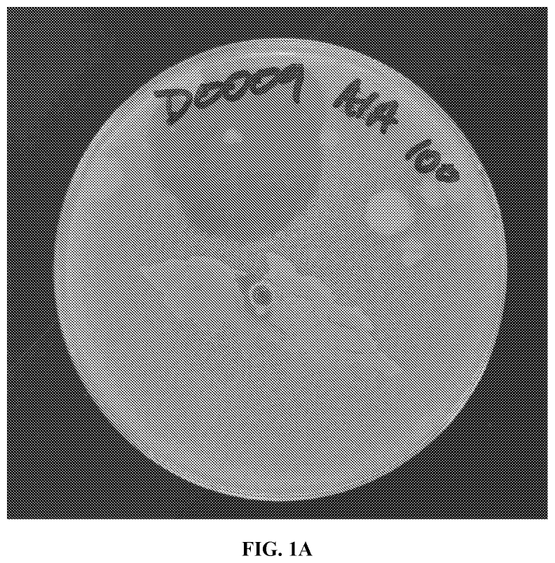

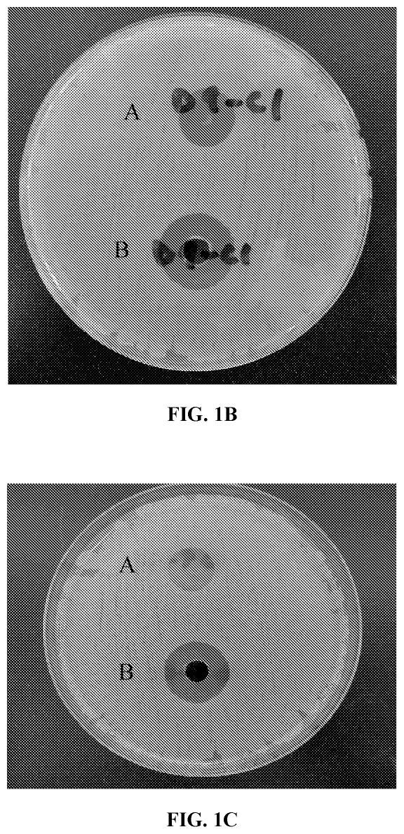

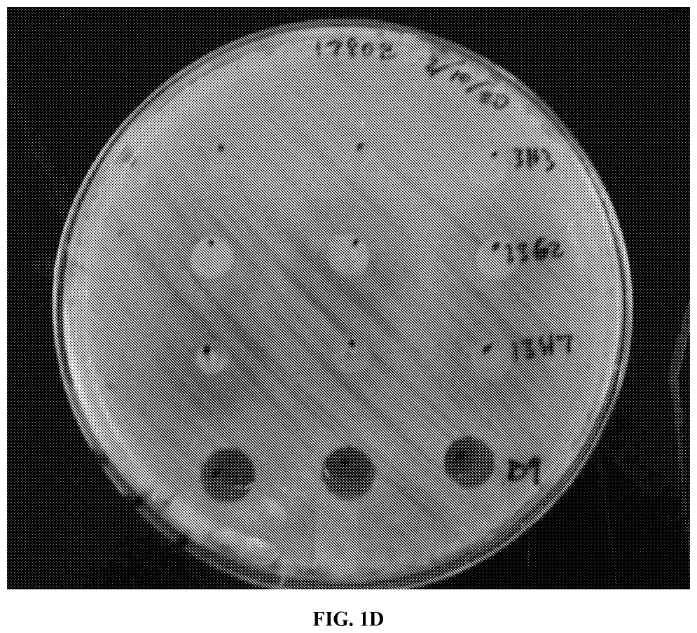

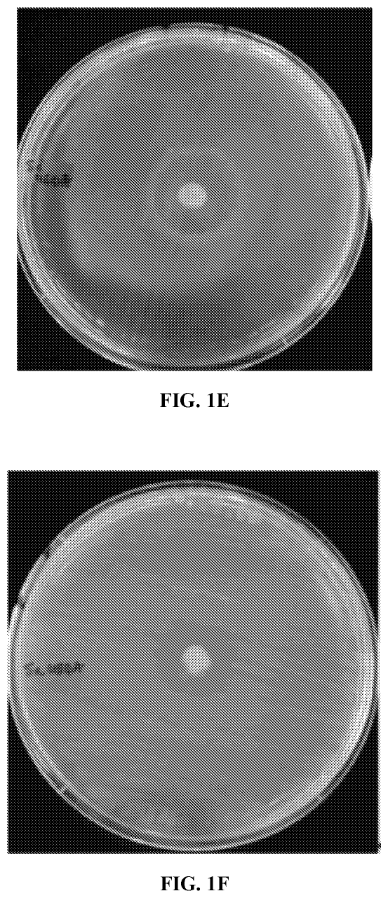

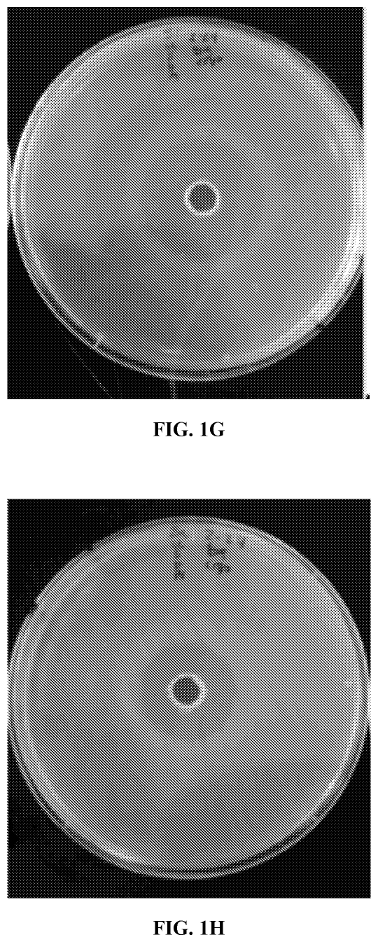

Overlay assays for discovery of PR-D9. To identify the environmental isolates with bio-activity against pathogens of interest, an overlay assay was performed. The overlay assay was performed using a strain of Vibrio parahaemolyticus (ATCC 17802). A colony of V. parahaemolyticus (ATCC 17802) was inoculated into 5 mL of liquid TSB+2-3% NaCl and grown at 220 rpm at 30° C. for ˜6-8 hours until an optical density of at least 1 was obtained. The liquid culture of V. parahaemolyticus was diluted into molten 55° C. TSA+2-3% NaCl agar to yield a final optical density of 0.005. Then, 5 mL of molten agar was poured or pipetted over the soil isolation plates and allowed to cool. The plates were then re-incubated at 30° C. overnight, and the following day colonies were checked for zones of clearance, indicating inhibition of the environmental isolate against Vibrio parahaemolyticus ATCC 17802. As shown in FIG. 1A, a mucoidal colony of a bacteria showed a large zone of inhibition against the target pathogen, Vibrio, and was subsequently isolated to purity and identified as PR-D9. This represents the first isolation of this strain from soil samples.

Spot-assay and agar well plug assay V. parahaemolyticus. To test the anti-pathogenic potential of PR-D9 secreted factors against V. parahaemolyticus, a combined spot-assay and agar plug assay was performed. Briefly, an agar plate covered in V. parahaemolyticus was exposed to a spot of whole fermentation broth of PR-D9, grown overnight at 30° C. at 220 RPM in TSB. Using a sterile borer, a well was bored into the same agar plate, which was then filled with cell-free supernatant from the same PR-D9 growth. As shown in FIGS. 1B and 1C both the broth and the cell-free supernatant treatments resulted in zones of inhibition against V. parahaemolyticus.

Comparison of PR-D9 and several other soil isolates of bacteria against V. parahaemolyticus growth. To test the efficacy of various soil isolates against V parahaemolyticus growth, an overnight challenge incubation was performed. Briefly, a culture of V. parahaemolyticus ATCC 17802 was grown overnight in saline TSB and a cotton swab was used to cover a TSA plate with a lawn of V. parahaemolyticus. 5 μL spots of whole fermentations of various soil isolates were plated in triplicate on the plate, and incubated overnight at 30° C. As shown in FIG. 1D, strain of interest, PR-D9 (labeled as “D9”) demonstrates anti-V. parahaemolyticus activity as shown by the zones of clearing.

Spot-assay and agar well plug assay Streptococcus iniae and Streptococcus agalactiae. To test the anti-pathogenic potential of PR-D9 secreted factors against S. iniae and S. agalactiae, spot-assays and agar plug assays were performed. Briefly, for the spot-assays, agar plates covered in S. iniae or S. agalactiae were exposed to a spot of whole fermentation broth of PR-D9, grown overnight at 30° C. at 220 RPM in TSB. For the well plug assays, using a sterile borer, a well was bored into agar plates covered in S. iniae or S. agalactiae, which were then filled with cell-free supernatant from the same PR-D9 growth. As shown in FIGS. 1E and 1F, strain of interest, PR-D9, demonstrates anti-S. iniae activity (FIG. 1E) and anti-S. agalactiae activity (FIG. 1F) as shown by the zones of clearing when 5 μL of strain PR-D9 whole fermentation broth was spotted on the agar plates. As shown in FIGS. 1G and 111, strain of interest, PR-D9, also demonstrates anti-S. iniae activity (FIG. 1G) and anti-S. agalactiae activity (FIG. 1H) as shown by the zones of clearing when 50 μL of strain PR-D9 cell-free supernatant was added to agar wells. Accordingly, these results demonstrate that both the broth, FIGS. 1E-1F, and the cell-free supernatant, FIGS. 1G-1H, treatments resulted in zones of inhibition against S. iniae and S. agalactiae.

Accordingly, these results demonstrate that the isolated PR-D9 strain of the present technology is effective in methods for inhibiting the growth of aquaculture pathogens.

### Example 2: The Paenibacillus Strain of the Present Technology Demonstrates Activity Against Vibrio parahaemolyticus

This example demonstrates that the Paenibacillus strain of the present technology, PR-D9, exhibits potent anti-V. Parahaemolyticus activity.

Methods. To test the efficacy of PR-D9 against V. parahaemolyticus growth in vitro, liquid broth and agar diffusion assays were carried out using a 1:2 dilution series of PR-D9 (as outlined in Table 4). Briefly, cryopreserved V. parahaemolyticus was thawed, streaked onto TSA2 and incubated at 30° C. for 22 hrs. A single colony was selected and incubated in 5 mL TSB2 for 18 hrs. Each experiment was completed in quadruplicate and included 100 μL bacterial stock and 100 μL of treatment in each well. The 10 dilutions of PR-D9 (Table 4), a positive control (“Pos Ctrl”) (media with bacteria and PBS), an LBF control (“LBF Blank”) (media without bacteria and neat PR-D9), and a media blank (media only and PBS) were tested.

TABLE 4
Stock and diluted concentrations of PR-D9.

| Dilution No. | Broth Assay | Agar Diffusion Assay |
|---|---|---|
| 1 | 12.50 | 25.00 |
| 2 | 6.25 | 12.50 |
| 3 | 3.13 | 6.25 |
| 4 | 1.56 | 3.13 |
| 5 | 0.78 | 1.56 |
| 6 | 0.39 | 0.78 |
| 7 | 0.20 | 0.39 |
| 8 | 0.10 | 0.20 |
| 9 | 0.05 | 0.10 |
| 10 | 0.025 | 0.05 |

Spectrophotometer readings (OD₆₀₀) were captured just prior to incubation and at 72 hours and are reported as delta OD₆₀₀ with media blank subtracted. An agar diffusion assay was also carried out for PR-D9, which was diluted as per Table 4 and 5 μL aliquots dispensed on TSA2 plates containing a lawn of V. parahaemolyticus. The dilution number was labelled on the bottom of each plate (n=4); all of which included a control (5 μL PBS). Plates were incubated for 24 hrs and each spot was monitored for zones of inhibition (no bacteria) or competition (growth of non-target bacteria).

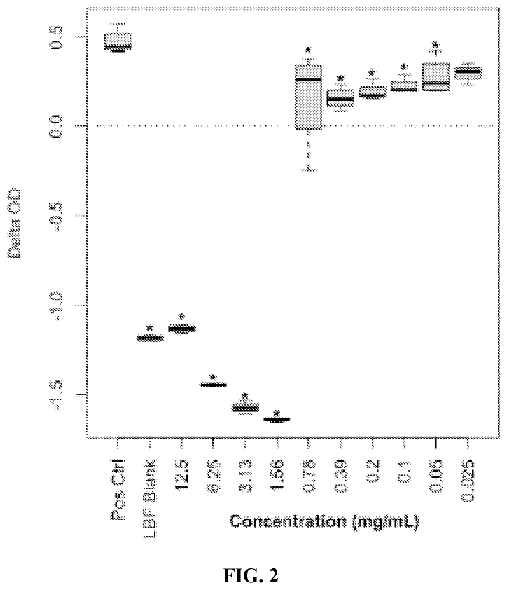

Results. As shown in FIG. 2, there was a significant inhibition of V. parahaemolyticus growth when treated with PR-D9 at concentrations from 0.05-0.78 mg/mL, as seen in the diminished Delta OD₆₀₀ values post incubation relative to the positive control, while PR-D9 concentrations in excess of 1.56 mg/mL neutralized V. parahaemolyticus, as seen in the negative Delta OD₆₀₀ values post incubation. Initial CFU/mL of bacterial stock was 4.7×10⁷. The OD₆₀₀ increased from 1.79 to 2.26 in controls through the 24 hr incubation. The liquid broth assay displayed significant reductions in Delta OD₆₀₀ values for concentrations of PR-D9 ranging from 12.5 mg/mL to 0.05 mg/mL when compared to positive controls. Notably, concentrations of 1.56 mg/mL and greater had significantly lower OD₆₀₀ readings compared with initial readings (i.e., negative delta OD₆₀₀; FIG. 2), suggesting neutralization of bacteria. This is supported by the LBF Blank sample (12.5 mg/mL LBF but no bacteria) having similar OD₆₀₀ values to the 12.5 mg/mL group (with bacteria; FIG. 2). Concentrations of PR-D9 between 0.05-0.78 mg/mL significantly inhibited bacterial growth compared to the positive control, while those ≤0.05 mg/mL had no significant effect (FIG. 2). The agar diffusion assay was less sensitive than the liquid broth assay, as determined by comparing PR-D9 across broth and plate assays. Treatment with ≥6.25 mg/mL PR-D9 resulted in inhibition between 4-10 mm in diameter for 3 of 4 replicates (data not shown).

Accordingly, these results show that the Paenibacillus strain of the present technology, PR-D9, exhibits potent anti-V. parahaemolyticus activity in vitro, and is effective in methods for reducing or inhibiting the growth of V. parahaemolyticus and in methods for preventing, reducing the risk of, or treating conditions associated with V. parahaemolyticus including, but not limited to, Acute Hepatopancreatic Necrosis Disease (AHPND), Early Mortality Syndrome (EMS), and White Fecal Disease (WFD).

### Example 3: The Paenibacillus Strain of the Present Technology Demonstrates Activity Against Streptococcus agalactiae

This example demonstrates that the Paenibacillus strain of the present technology, PR-D9, exhibits potent anti-S. agalactiae activity.

Methods. To test the efficacy of PR-D9 against S. agalactiae growth in vitro, liquid broth and agar diffusion assays were carried out using a 1:2 dilution series of PR-D9 (as outlined in Table 4). Briefly, cryopreserved S. agalactiae was thawed, streaked onto TSAY and incubated at 28° C. for 4 days. A single colony was selected and incubated in 3 mL TSBY for 48 hrs. Each group was completed in quadruplicate and included 100 μL bacterial stock and 100 μL of treatment in each well. The 10 dilutions of PR-D9 were carried out as shown in Table 4, a positive control (media with bacteria and PBS), an LBF control (media without bacteria and neat PR-D9), and a media blank (media only and PBS) were tested. Spectrophotometer readings (OD₆₀₀) were captured just prior to incubation and at 96 hours and are reported as Delta OD₆₀₀ with media blank subtracted. An agar diffusion assay was also carried out, which were diluted as per Table 4, and 5 μL aliquots dispensed on TSAY plates containing a lawn of S. agalactiae. The dilution number was labelled on the bottom of each plate (n=4). Plates were incubated for 50 hrs and each spot was monitored for zones of inhibition (no bacteria) or competition (growth of non-target bacteria).

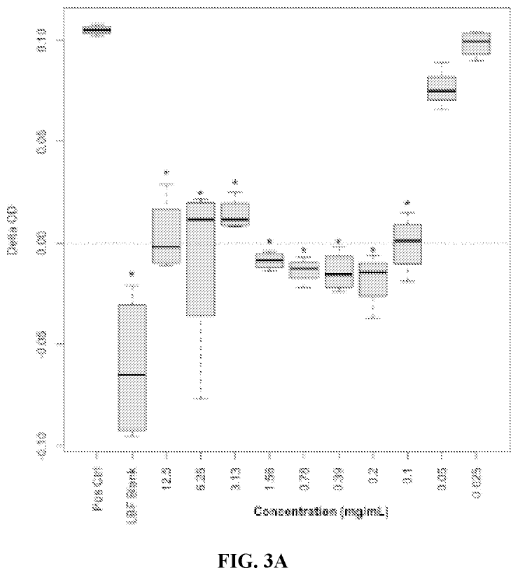

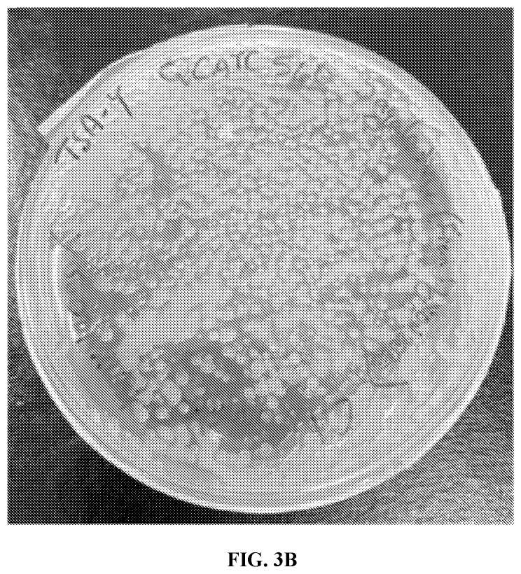

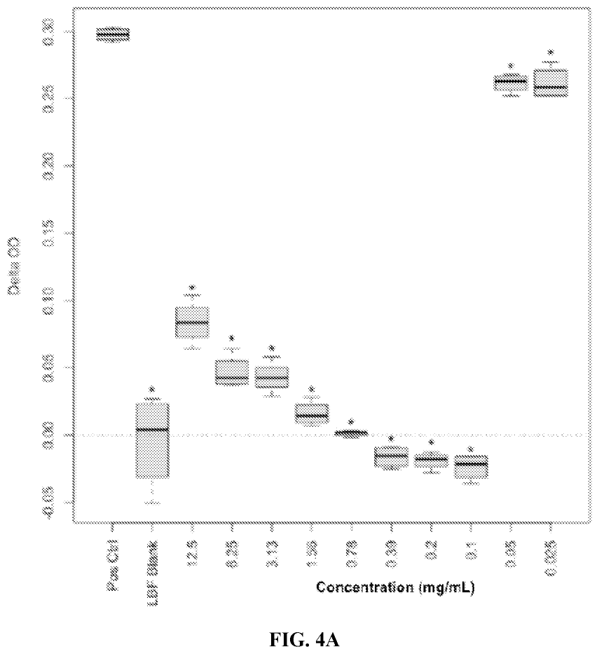

Results. As shown in FIG. 3A, there was a significant inhibition of S. agalactiae growth when treated with PR-D9 at concentrations from 0.1-12.5 mg/mL, as seen in the diminished Delta OD₆₀₀ values post incubation relative to the positive control. The concentration of bacteria present in each well at the start of the liquid broth assay was 4.7×10⁸ CFU/mL. The OD₆₀₀ of positive controls increased from 0.12 to 0.23 through the 96 hr incubation. Significantly lower OD₆₀₀ values were observed in 12.5 mg/mL to 0.1 mg/mL PR-D9 compared with controls (FIG. 3A). Inhibition of S. agalactiae was similar for concentrations ≥0.1, which showed average delta OD₆₀₀ values near 0, with an efficacy threshold at 0.1 mg/mL. At 12.5 mg/mL PR-D9, bacteria were inhibited, but not neutralized (Delta OD₆₀₀ approximately 0; FIG. 3A), which was further supported by plating of 100 μL of the 12.5 mg/mL group on TSAY resulting in a lawn of S. agalactiae (FIG. 3B).

Accordingly, these results show that the Paenibacillus strain of the present technology, PR-D9, exhibits potent anti-S. agalactiae activity, and is effective in methods for reducing or inhibiting the growth of S. agalactiae and in methods for preventing, reducing the risk of, or treating conditions associated with S. agalaciae including, but not limited to, meningitis.

### Example 4: The Paenibacillus Strain of the Present Technology Demonstrates Activity Against Streptococcus iniae

This example demonstrates that the Paenibacillus strain of the present technology, PR-D9, exhibits potent anti-S. iniae activity.

Methods. To test the efficacy of PR-D9 against S. iniae growth in vitro, liquid broth and agar diffusion assays were carried out using a 1:2 dilution series of PR-D9 (as outlined in Table 4). Briefly, cryopreserved S. iniae was thawed, streaked onto TSAY and incubated at 28° C. for 4 days. A single colony was selected and incubated in 3 mL TSBY for 48 hrs. Each experiment was completed in quadruplicate and included 100 μL bacterial stock and 100 μL of treatment in each well. The 10 dilutions of PR-D9 (Table 4), a positive control (media with bacteria and PBS), an LBF control (media without bacteria and neat PR-D9), and a media blank (media only and PBS) were tested. Spectrophotometer readings (OD₆₀₀) were captured just prior to incubation and at 24 hours and are reported as delta OD₆₀₀ with media blank subtracted. An agar diffusion assay was also carried out for PR-D9, which was diluted as per Table 4 and 5 μL aliquots dispensed on TSAY plates containing a lawn of S. iniae. The dilution number was labelled on the bottom of each plate (n=4); all of which included a control (5 μL PBS). Plates were incubated for 24 hrs and each spot was monitored for zones of inhibition (no bacteria) or competition (growth of non-target bacteria).

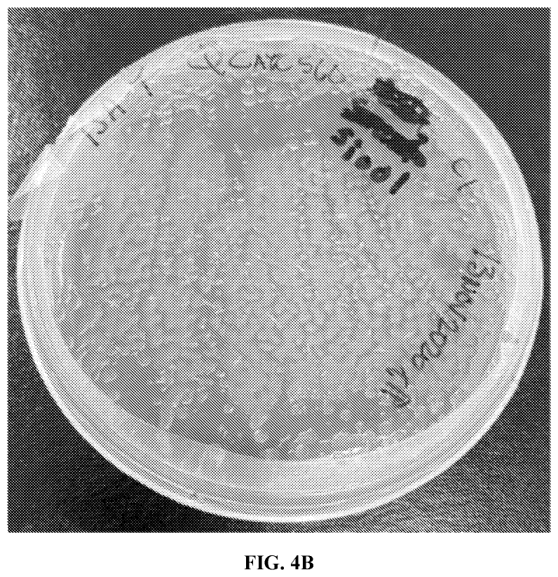

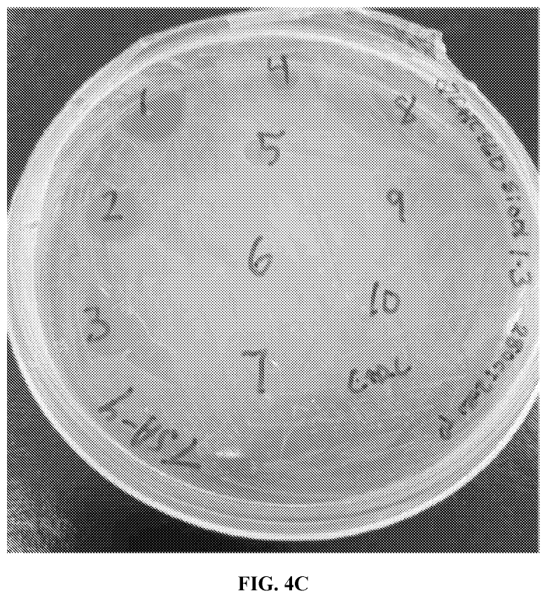

Results. As shown in FIG. 4A, there was a significant inhibition of S. iniae growth when treated with PR-D9 at concentrations from 0.1-12.5 mg/mL, as seen in the diminished Delta OD₆₀₀ values post incubation relative to the positive control. Initial concentrations of S. iniae were 5.8×10⁷ CFU/mL. The OD₆₀₀ increased from 0.21 to 0.50 in controls through the 24 hr incubation. The liquid broth assay displayed significant reductions in OD₆₀₀ values for concentrations of PR-D9 ranging from 12.5 mg/mL to 0.025 mg/mL when compared to positive controls. The inhibition of growth was not complete at 12.5 mg/mL as positive delta OD₆₀₀ values were observed (FIG. 4A). However, a linear decrease in delta OD₆₀₀ values was observed from 12.5-0.1 mg/mL, where negative OD₆₀₀ values were observed at 0.1 mg/mL PR-D9. A typical hypothesis would be that PR-D9 grew during the incubation time; however, this seems unlikely given the PR-D9 Blank Control had average delta OD₆₀₀ values of 0. Although concentrations of ≤0.05 mg/mL PR-D9 had significantly lower delta OD₆₀₀ values compared to controls, the difference was slight and a clear threshold of efficacy exists at 0.1 mg/mL PR-D9 (FIG. 4A). Plating of 100 μL of 12.5 mg/mL PR-D9 treated S. iniae resulted in a lawn of bacteria, confirming that treated bacteria have the capacity to grow on agar post-treatment in broth (FIG. 4B). As shown by FIG. 4C, growth of S. iniae was inhibited by treatment with ≥1.56 mg/mL PR-D9 in an agar diffusion assay.

Accordingly, these results show that the Paenibacillus strain of the present technology, PR-D9, exhibits potent anti-S. iniae activity and is effective in methods for reducing or inhibiting the growth of S. iniae and in methods for preventing, reducing the risk of, or treating conditions associated with S. iniae including, but not limited to, meningitis.

### Example 5: The Paenibacillus Strain of the Present Technology Demonstrates Activity Against Aeromonas salmonicida

This example demonstrates that the Paenibacillus strain of the present technology, PR-D9, exhibits potent anti-A. salmonicida activity.

Methods. To test the efficacy of PR-D9 against A. salmonicida growth in vitro, liquid broth and agar diffusion assays were carried out using a 1:2 dilution series of PR-D9 (as outlined in Table 4). Briefly, cryopreserved A. salmonicida (AS001) was thawed, streaked onto TSA and incubated at 20° C. for 5 days. A single colony was selected and incubated in 3 mL TSB for 22 hrs with continuous shaking at 200 rpm. Each experiment was completed in quadruplicate and included 100 μL bacterial stock and 100 μL of treatment in each well. The 10 dilutions of PR-D9 (Table 4), a positive control (media with bacteria and PBS), an LBF control (media without bacteria and neat PR-D9), and a media blank (media only and PBS) were tested. Spectrophotometer readings (OD₆₀₀) were captured just prior to incubation and at 48 hours and are reported as delta OD₆₀₀ with media blank subtracted. An agar diffusion assay was also carried out for PR-D9, which was diluted as per Table 4 and 5 μL aliquots dispensed on TSA media plates containing a lawn of A. salmonicida. The dilution number was labelled on the bottom of each plate (n=4); which included a control (5 μL PBS). Plates were incubated for 24 hrs and each spot was monitored for zones of inhibition (no bacteria) or competition (growth of non-target bacteria).

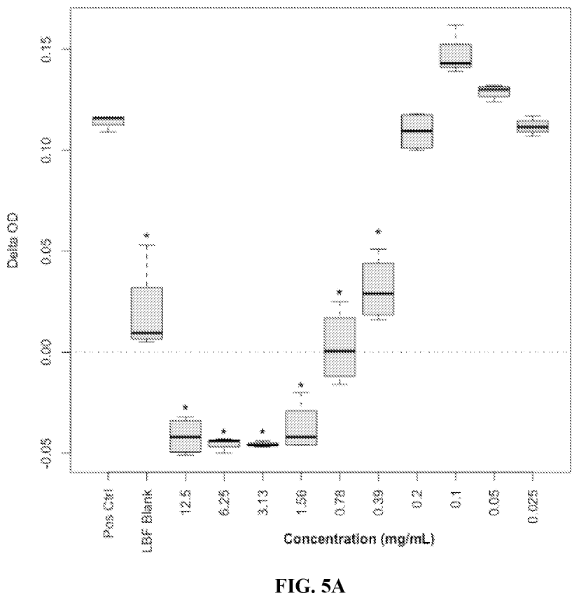

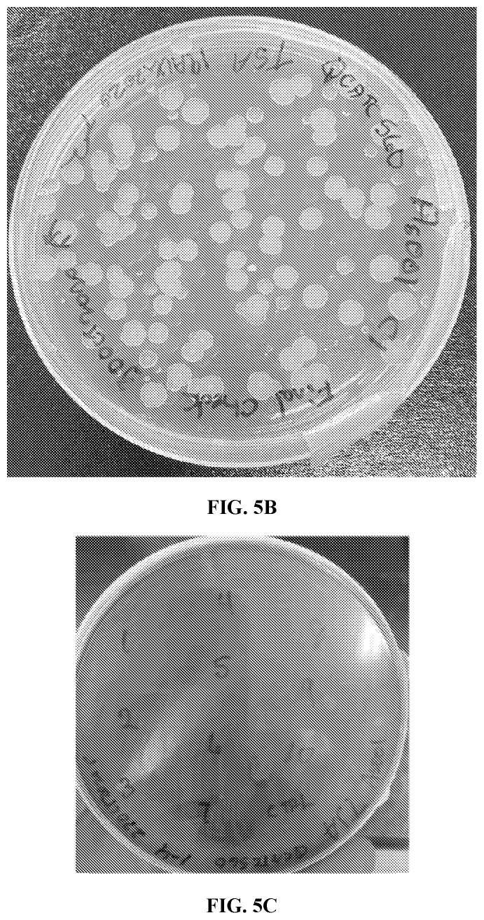

Results. As shown in FIG. 5A, there was a significant inhibition of A. salmonicida growth when treated with PR-D9 at concentrations from 0.39-0.78 mg/mL, as seen in the diminished Delta OD₆₀₀ values post incubation relative to the positive control, while PR-D9 concentrations in excess of 1.56 mg/mL neutralized A. salmonicida, as seen in the negative Delta OD₆₀₀ values post incubation. The concentration of bacteria present in each well at the start of the liquid broth assay was 3.6×108 CFU/mL. The OD₆₀₀ of positive controls increased from 0.067 to 1.81 through the 48 hr incubation. Significantly lower OD₆₀₀ values were observed in 0.39 mg/mL PR-D9 compared with controls (FIG. 5A). Concentrations ≥1.56 mg/mL had negative delta OD₆₀₀ values, suggesting neutralization. Plating of 100 μL of the 12.5 mg/mL group on TSA resulted in approximately 125 colonies after 48 hrs of incubation (FIG. 5B). As shown in FIG. 5C, growth of A. salmonicida was not inhibited by PR-D9; however, white spotted growth was observed up to 6.25 mg/mL of PR-D9, indicating potential PR-D9 satellite colony growth (FIG. 5C).

Accordingly, overall, these results show that the Paenibacillus strain of the present technology, PR-D9, exhibits potent anti-A. salmonicida activity and is effective in methods for reducing or inhibiting the growth of A. salmonicida and in methods for preventing, reducing the risk of, or treating conditions associated with A. salmonicida including, but not limited to, furunculosis.

### Example 6: The Paenibacillus Strain of the Present Technology Demonstrates Activity Against Flavobacterium psychrophilum

This example demonstrates that the Paenibacillus strain of the present technology, PR-D9, exhibits potent anti-F. psychrophilium activity.

Methods. To test the efficacy of PR-D9 against F. psychrophilium growth in vitro, liquid broth and agar diffusion assays were carried out using a 1:2 dilution series of PR-D9 (as outlined in Table 4). Briefly, cryopreserved F. psychrophilum bacteria were streaked onto appropriate media and incubated (TYES for 7 days at 15° C.), then grown in broth (3 mL TYES for 5 days at 15° C.). Equal volumes of F. psychrophilium and the appropriate PR-D9 dilution were mixed and an initial optical density point was taken. After 7 days another reading was taken to measure changes in growth.

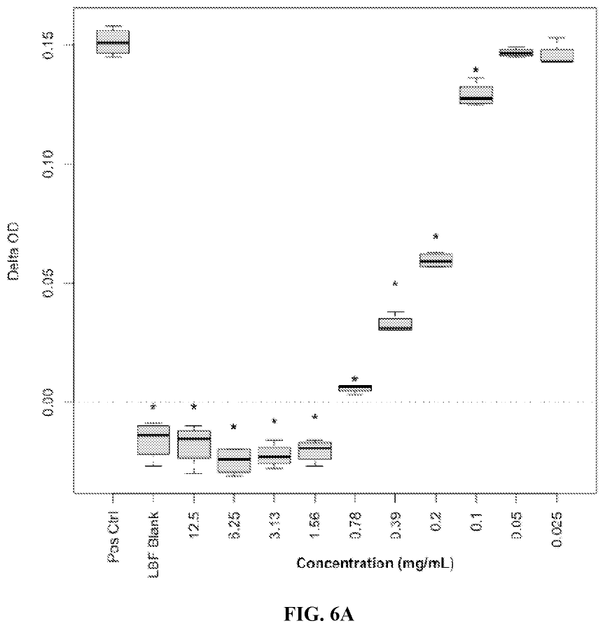

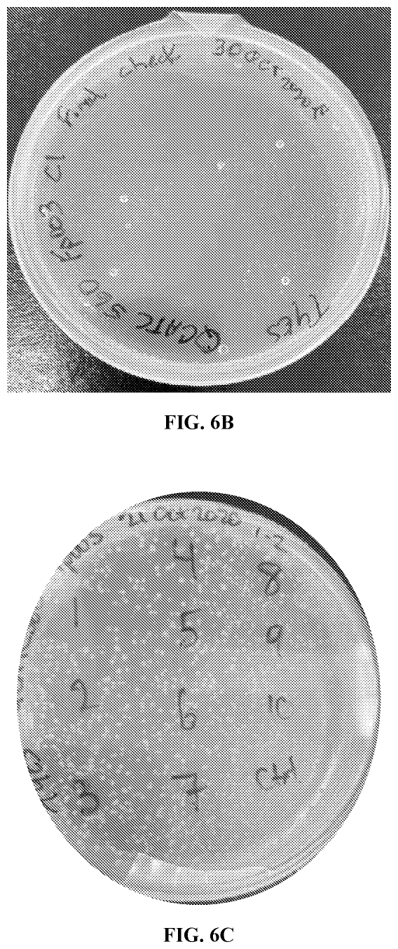

Results. As shown in FIG. 6A, there was a significant inhibition of F. psychrophilium growth when treated with PR-D9 at concentrations from 0.1-12.5 mg/mL, as seen in the diminished Delta OD₆₀₀ values post incubation relative to the positive control. The concentration of bacteria present in each well at the start of the liquid broth assay was 7.7×10⁷ CFU/mL. The OD₆₀₀ of positive controls increased from 0.070 to 0.22 through the 7 day incubation. Significantly lower OD₆₀₀ values were observed in ≥0.1 mg/mL PR-D9 compared with controls (FIG. 6A). The rate of F. psychrophilum inhibition followed a classical dose-response curve, capturing the minimum asymptote (≤0.05 mg/mL), exponential slope (0.78-0.10 mg/mL), and maximum asymptote (i.e., maximal efficacy; ≥1.56 mg/mL). At 12.5 mg/mL PR-D9, bacteria were inhibited, but not neutralized (delta OD₆₀₀ approximately 0), which was further supported by plating of 100 μL of the 12.5 mg/mL group on TYES media resulting in a lawn of F. psychrophilum (FIG. 6B). As shown by FIG. 6C, growth of F. psychrophilium was inhibited by PR-D9 in an agar diffusion assay. Treatments ≥1.56 mg/mL PR-D9 resulted in inhibition in all replicates. Areas of inhibition measuring 6.5 mm in diameter were observed at the highest concentrations.

Accordingly, these results show that the Paenibacillus strain of the present technology, PR-D9, exhibits potent anti-F. psychrophilium activity and is effective in methods for reducing or inhibiting the growth of F. psychrophilium and in methods for preventing, reducing the risk of, or treating conditions associated with F. psychrophilium including, but not limited to, bacterial cold water disease (BCWD).

### Example 7: The Paenibacillus Strain of the Present Technology Demonstrates Activity Against Photobacterium damselae subsp. piscida

This example demonstrates that the Paenibacillus strain of the present technology, PR-D9, exhibits potent anti-P. damselae subsp. piscida activity.

Methods. To test the efficacy of PR-D9 against P. damselae subsp. piscida growth in vitro, liquid broth and agar diffusion assays were carried out using a 1:2 dilution series of PR-D9 (as outlined in Table 4). Briefly, cryopreserved P. damselae subsp. piscida bacteria were streaked onto appropriate media and incubated (TSA2 for 48 hours at 25° C.), then grown in broth (10 mL TSB2 for 18 hours at 150 RPM at 25° C.). Equal volumes of P. damselae subsp. piscida and the appropriate PR-D9 dilution were mixed and an initial optical density point was taken. After 24 hours another reading was taken to measure changes in growth.

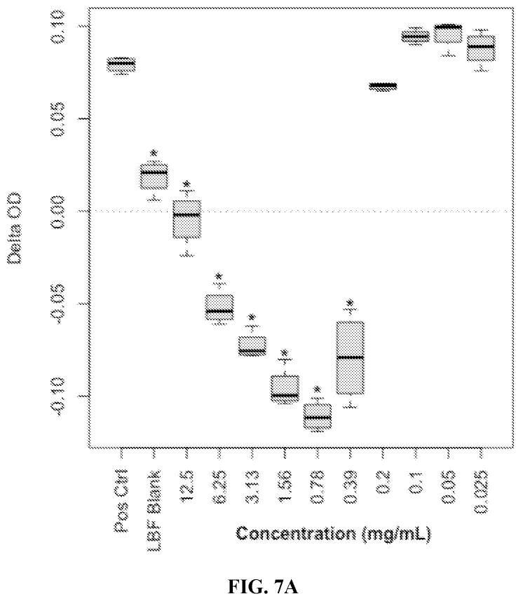

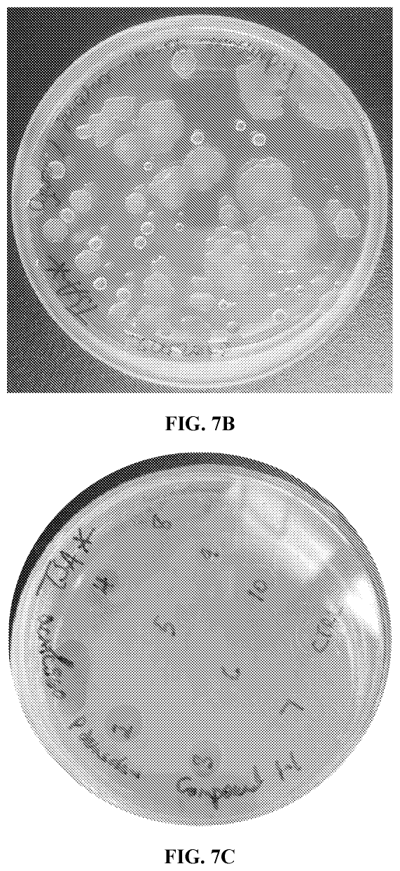

Results. As shown in FIG. 7A, there was neutralization of P. damselae subsp. piscida when treated with PR-D9 at concentrations from 0.39-12.5 mg/mL, as seen in the negative Delta OD₆₀₀ values post incubation. Initial CFU/mL of bacterial stock was 2.7×10⁸. The OD₆₀₀ increased from 0.37 to 0.53 in controls through the 7 hr incubation. The liquid broth assay displayed significant reductions in OD₆₀₀ values for concentrations of PR-D9 ranging from 12.5 mg/mL to 0.39 mg/mL when compared to positive controls; all of which had significantly lower OD₆₀₀ readings compared with initial readings of bacterial stock (FIG. 7A), suggesting neutralization of the bacteria. Broth was removed from the undiluted treatment group (12.5 mg/mL PR-D9) and was plated neat. Bacterial growth on this plate after 24 hrs did not present as P. damselae subsp. piscida, suggesting non-target growth, possibly of PR-D9 (FIG. 7B). Concentrations of PR-D9≤0.2 mg/mL had no significant effect compared to control (FIG. 7A). As shown by FIG. 7C, growth of P. damselae subsp. piscida was inhibited by PR-D9 at concentrations ≥3.13 mg/mL in an agar diffusion assay, with areas of inhibition measuring between 6 and 11 mm in diameter.

Accordingly, these results show that the Paenibacillus strain of the present technology, PR-D9, exhibits potent anti-P. damselae subsp. piscida activity and is effective in methods for reducing or inhibiting the growth of P. damselae subsp. piscida and in methods for preventing, reducing the risk of, or treating conditions associated with P. damselae subsp. piscida including, but not limited to, photobacteriosis.

### Example 8: The Paenibacillus Strain of the Present Technology Demonstrates Activity Against Vibrio anguillarum

This example demonstrates that the Paenibacillus strain of the present technology, PR-D9, exhibits potent anti-V. anguillarum activity.

Methods. To test the efficacy of PR-D9 against V. anguillarum growth in vitro, liquid broth and agar diffusion assays were carried out using a 1:2 dilution series of PR-D9 (as outlined in Table 4). Briefly, cryopreserved V. anguillarum bacteria were streaked onto appropriate media and incubated (TSA for 48 hours at 20° C.), then grown in broth (3 mL TSB for 22 hours at 200 RPM at 20° C.). Equal volumes of V. anguillarum and the appropriate PR-D9 dilution were mixed and an initial optical density point was taken.

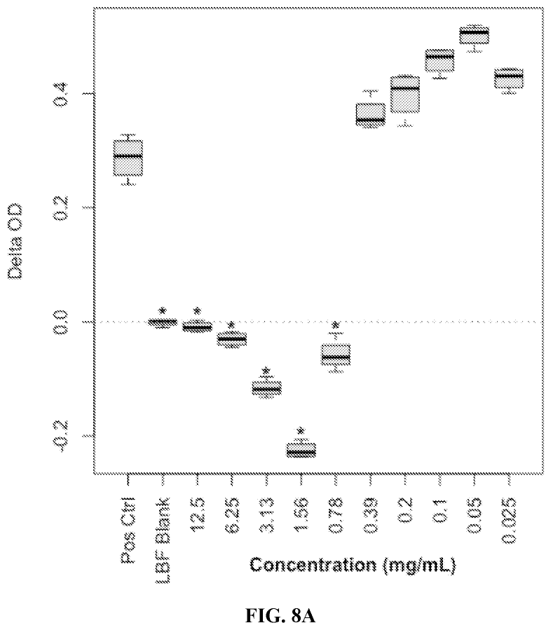

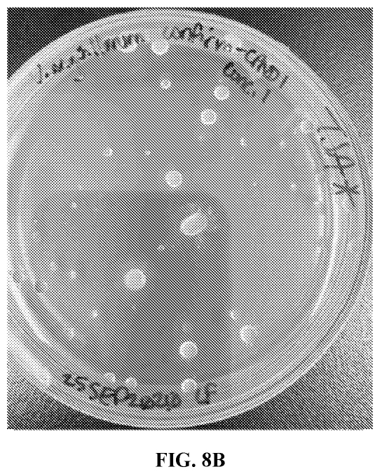

Results. After 24 hours another reading was taken to measure changes in growth. As shown in FIG. 8A, there was inhibition of V. anguillarum when treated with PR-D9 at concentrations from 0.78-12.5 mg/mL, as seen in the diminished Delta OD₆₀₀ values post incubation relative to the positive control. The concentration of bacteria present in each well at the start of the liquid broth assay was 1.4×10⁸ CFU/mL. The OD₆₀₀ of positive controls increased from 0.64 to 1.22 through the 24 hr incubation, with significantly lower OD₆₀₀ values observed in ≥0.78 mg/mL PR-D9 compared with controls (FIG. 8A). In all of these groups, OD₆₀₀ readings were lower than initial readings prior to incubation, suggesting neutralization of some bacteria. Broth removed from the undiluted treatment group (12.5 mg/mL PR-D9) and plated resulted in the growth of approximately 410 CFU/mL (FIG. 8B). Concentrations of PR-D9≤0.39 mg/mL had no significant effect compared to control, and in some cases (e.g., 0.05 mg/mL PR-D9), had significantly higher OD₆₀₀ values relative to control (FIG. 8A).

Accordingly, overall, these results show that the Paenibacillus strain of the present technology, PR-D9, exhibits potent anti-V. anguillarum activity and is effective in methods for reducing or inhibiting the growth of V. anguillarum and in methods for preventing, reducing the risk of, or treating conditions associated with V. anguillarum including, but not limited to, vibriosis, EMS, AHPND, and WFD.

### Example 9: The Paenibacillus Strain of the Present Technology Demonstrates Activity Against Yersinia ruckeri

This example demonstrates that the Paenibacillus strain of the present technology, PR-D9, exhibits potent anti-Y. ruckeri activity.

Methods. To test the efficacy of PR-D9 against Y. ruckeri growth in vitro, liquid broth and agar diffusion assays were carried out using a 1:2 dilution series of PR-D9 (as outlined in Table 4). Briefly, cryopreserved Y. ruckeri bacteria were streaked onto appropriate media and incubated (TSA for 48 hours at 20° C.), then grown in broth (3 mL TSB for 22 hours at 20° C.). Equal volumes of Y. ruckeri and the appropriate PR-D9 dilution were mixed and an initial optical density point was taken. After 72 hours another reading was taken to measure changes in growth.

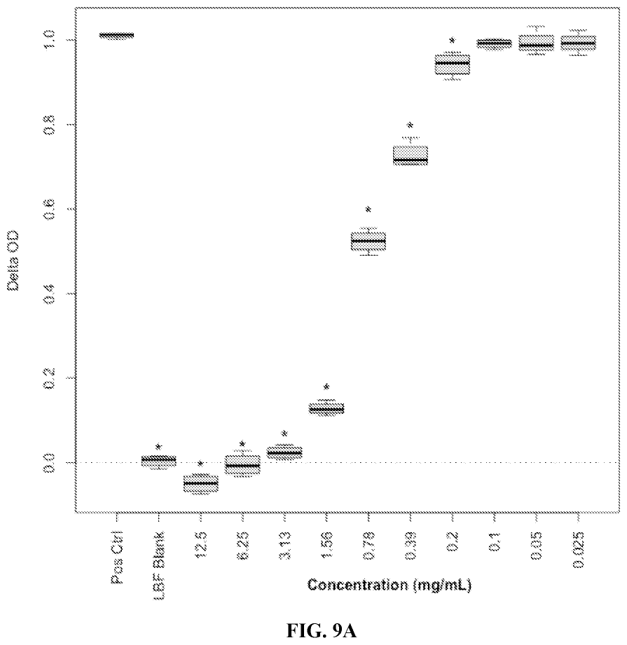

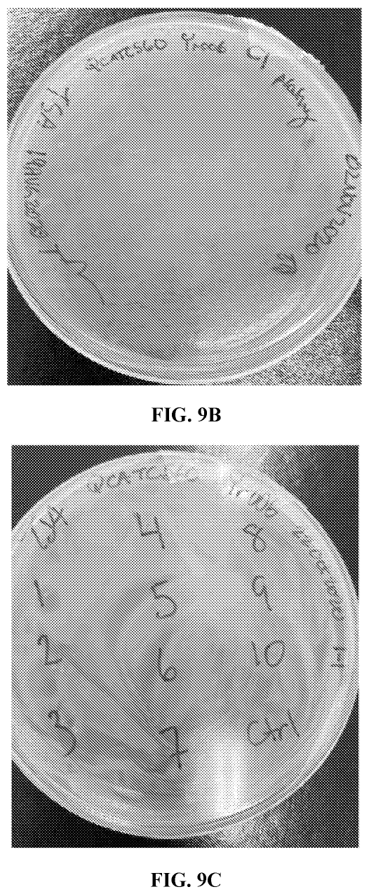

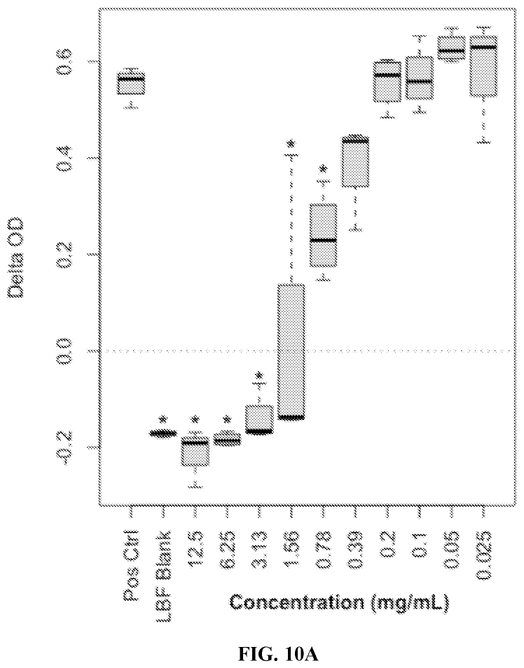

Results. As shown in FIG. 9A, there was inhibition of Y. ruckeri when treated with PR-D9 at concentrations from 0.2-12.5 mg/mL, as seen in the diminished Delta OD₆₀₀ values post incubation relative to the positive control. The concentration of bacteria present in each well at the start of the liquid broth assay was 1.4×10⁸ CFU/mL. The OD₆₀₀ of positive controls increased from 0.20 to 1.21 through the 72 hr incubation. Significantly lower OD₆₀₀ values were observed in 12.5 mg/mL to 0.2 mg/mL PR-D9 compared with controls (FIG. 9A). The rate of Y. ruckeri inhibition followed a classical dose-response curve, capturing the minimum asymptote (0.1-0.025 mg/mL), exponential slope (1.56-0.2 mg/mL), and maximum asymptote (i.e., maximal efficacy; 12.50-3.13 mg/mL). At 12.5 mg/mL PR-D9, bacteria were inhibited, but not neutralized (delta OD approximately 0), which was further supported by plating of 100 μL of the 12.5 mg/mL group on TSA media resulting in a lawn of Y. ruckeri (FIG. 9B). As shown by FIG. 9C, growth of Y. ruckeri was inhibited by PR-D9 at a concentration of 25 mg/mL in an agar diffusion assay, with areas of inhibition measuring between 3 mm in diameter. White spotted growth was observed alongside Y. ruckeri at concentrations ≥3.13 mg/mL PR-D9, indicating potential PR-D9 satellite colony growth.

Accordingly, these results show that the Paenibacillus strain of the present technology, PR-D9, exhibits potent anti-Y. ruckeri activity and is effective in methods for reducing or inhibiting the growth of Y. ruckeri and in methods for preventing, reducing the risk of, or treating conditions associated with Y. ruckeri including, but not limited to, enteric redmouth (ERM) disease.

### Example 10: The Paenibacillus Strain of the Present Technology Demonstrates Activity Against Saprolegnia parasitica

This example demonstrates that the Paenibacillus strain of the present technology, PR-D9, exhibits potent anti-S. parasitica activity.

Methods. To test the efficacy of PR-D9 against S. parasitica growth in vitro, liquid broth and agar diffusion assays were carried out using a 1:2 dilution series of PR-D9 (as outlined in Table 4). Briefly, cryopreserved S. parasitica were streaked onto appropriate media and incubated (GY for 6 days at 20° C.), then grown in broth (45 mL GY for 5 days at 20° C.). Equal volumes of S. parasitica and the appropriate PR-D9 dilution were mixed and an initial optical density point was taken. After 96 hours another reading was taken to measure changes in growth.

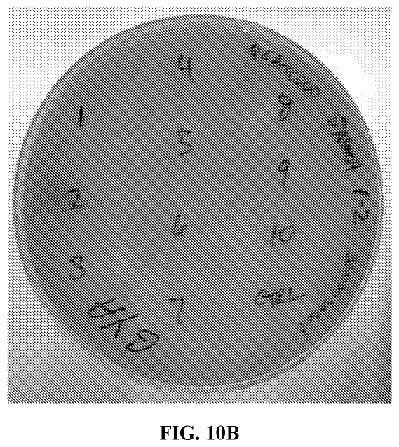

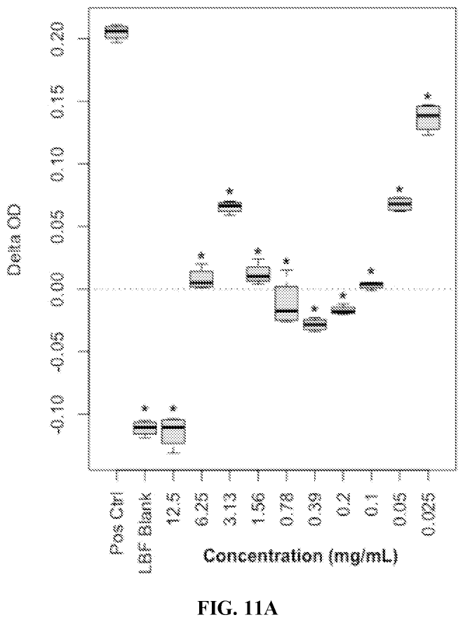

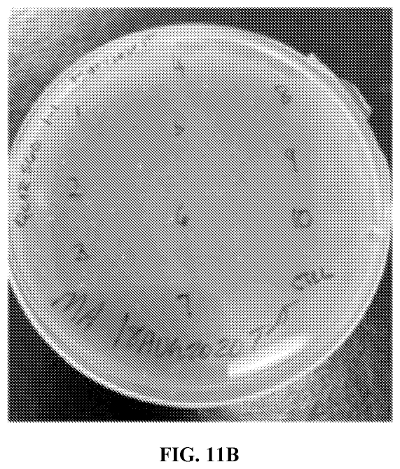

Results. As shown in FIG. 10A, there was inhibition of S. parasitica when treated with PR-D9 at concentrations from 0.78-1.56 mg/mL. The concentration of S. parasitica present in each well at the start of the liquid broth assay was difficult to determine using SSC counts given diffuse growth of mycelia. Thus, initial concentrations of S. parasitica were not quantifiable. The OD₆₀₀ of positive controls increased from 0.060 to 0.61 through the 96 hr incubation. Significantly lower OD₆₀₀ values were observed in 12.5 mg/mL to 0.78 mg/mL PR-D9 compared with controls (FIG. 10A). The rate of S. parasitica inhibition followed a classical dose-response curve, capturing the minimum asymptote (0.2-0.025 mg/mL), exponential slope (1.56-0.39 mg/mL), and maximum asymptote (i.e., maximal efficacy; 12.5-3.13 mg/mL). At concentrations ≥3.13 mg/mL PR-D9, the oomycete showed signs of being neutralized based on negative delta OD values. As shown in FIG. 10B, growth of S. parasitica was not inhibited by PR-D9.

Accordingly, overall, these results show that the Paenibacillus strain of the present technology, PR-D9, exhibits potent anti-S. parasitica activity and is effective in methods for reducing or inhibiting the growth of S. parasitica and in methods for preventing, reducing the risk of, or treating conditions associated with S. parasitica.

### Example 11: The Paenibacillus Strain of the Present Technology Demonstrates Activity Against Tenacibaculum maritimum

This example demonstrates that the Paenibacillus strain of the present technology, PR-D9, exhibits potent anti-T. maritimum activity.

Methods. To test the efficacy of PR-D9 against S. parasitica growth in vitro, liquid broth and agar diffusion assays were carried out using a 1:2 dilution series of PR-D9 (as outlined in Table 4). Briefly, cryopreserved T. maritimum were streaked onto appropriate media and incubated (TSA for 7 days at 15° C.), then grown in broth (10 mL TSB for 7 days at 15° C.). 180p1 T. maritimum and 20 μl of the appropriate PR-D9 dilution were mixed and an initial optical density point was taken. After 7 days another reading was taken to measure changes in growth.

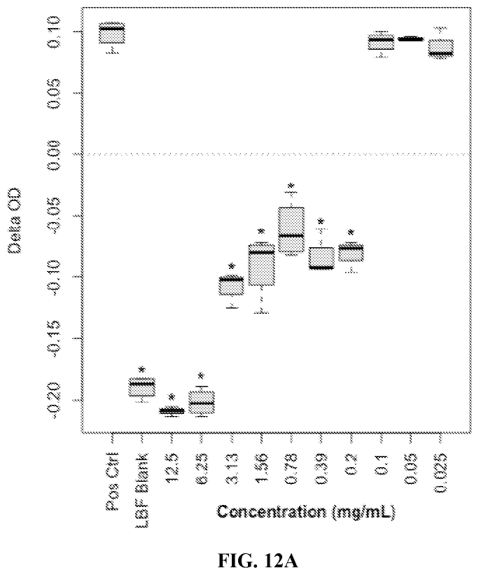

Results. As shown in FIG. 11A, there was inhibition of T. maritimum when treated with PR-D9 at concentrations from 0.025-12.5 mg/mL, as seen in the diminished Delta OD₆₀₀ values post incubation relative to the positive control. As shown in FIG. 11B, the highest concentrations for PR-D9 (≥6.25 mg/mL) resulted in growth inhibition in all replicates, with zones of inhibition of 0.5-2 mm.

Accordingly, these results show that the Paenibacillus strain of the present technology, PR-D9, exhibits potent anti-T. maritimum activity and is effective in methods for reducing or inhibiting the growth of T. maritimum and in methods for preventing, reducing the risk of, or treating conditions associated with T. maritimum including, but not limited to, tenacibaculosis.

### Example 12: The Paenibacillus Strain of the Present Technology Demonstrates Activity Against Moritella viscosa

This example demonstrates that the Paenibacillus strain of the present technology, PR-D9, exhibits potent anti-M. viscosa activity.

Methods. To test the efficacy of PR-D9 against M. viscosa growth in vitro, liquid broth and agar diffusion assays were carried out using a 1:2 dilution series of PR-D9 (as outlined in Table 4). Briefly, cryopreserved M. viscosa were streaked onto appropriate media and incubated (TSA2 for 5 days at 15° C.), then grown in broth (3 mL TSB2 for 48 hours at 200 RPM at 15° C.). 180 μl M. viscosa and 20 μl of the appropriate PR-D9 dilution were mixed and an initial optical density point was taken. After 96 hours another reading was taken to measure changes in growth.

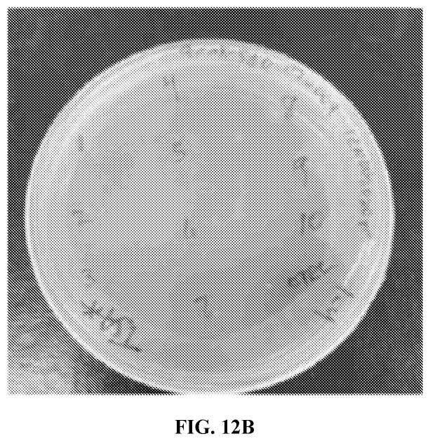

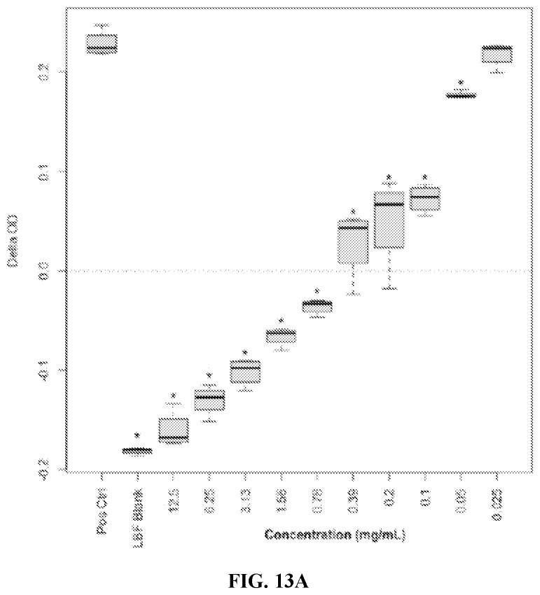

Results. As shown in FIG. 12A, there was inhibition of M. viscosa when treated with PR-D9 at concentrations from 0.2-12.5 mg/mL, as seen in the diminished Delta OD₆₀₀ values post incubation relative to the positive control. The concentration of bacteria present in each well at the start of the liquid broth assay was 2.2×10⁸ CFU/mL. The OD₆₀₀ of positive controls increased from 0.21 to 0.31 through the 96 hr incubation. Significantly lower OD₆₀₀ values were observed in concentrations ≥0.20 mg/mL PR-D9 compared with controls; all of which had negative delta OD values (FIG. 12A). Separation between concentrations 6.25-3.13 mg/mL and 0.2-0.1 mg/mL suggests thresholds of efficacy between these concentrations. As shown in FIG. 12B, growth of M. viscosa was not inhibited by PR-D9. However, white spotted growth was observed up to 6.25 mg/mL of PR-D9 (FIG. 12B), indicating potential PR-D9 satellite growth.

Accordingly, overall, these results show that the Paenibacillus strain of the present technology, PR-D9, exhibits potent anti-M. viscosa activity and is effective in methods for reducing or inhibiting the growth of M. viscosa and in methods for preventing, reducing the risk of, or treating conditions associated with M. viscosa including, but not limited to, winter ulcer disease.

### Example 13: The Paenibacillus Strain of the Present Technology Demonstrates Activity Against Flavobacterium columnare

This example demonstrates that the Paenibacillus strain of the present technology, PR-D9, exhibits potent anti-F. columnare activity.

Methods. To test the efficacy of PR-D9 against F. columnare growth in vitro, liquid broth and agar diffusion assays were carried out using a 1:2 dilution series of PR-D9 (as outlined in Table 4). Briefly, cryopreserved F. columnare were streaked onto appropriate media and incubated (TYES for 4 days at 25° C.), then grown in broth (10 mL TYES for 72 hours 25° C.). Equal volumes of F. columnare and of the appropriate PR-D9 dilution were mixed and an initial optical density point was taken. After 72 hours another reading was taken to measure changes in growth.

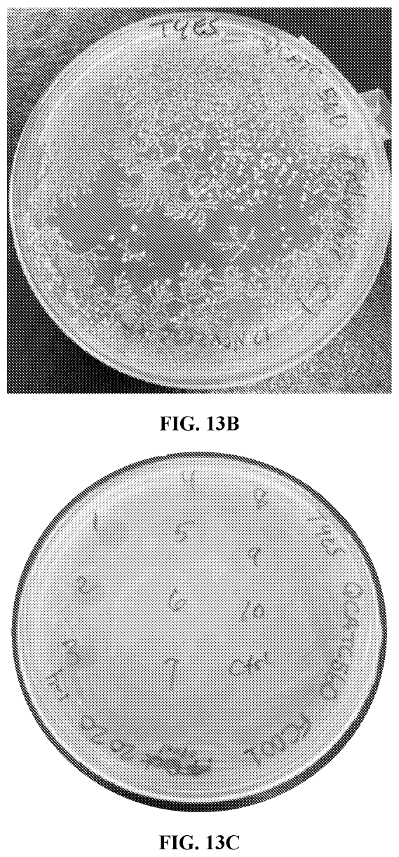

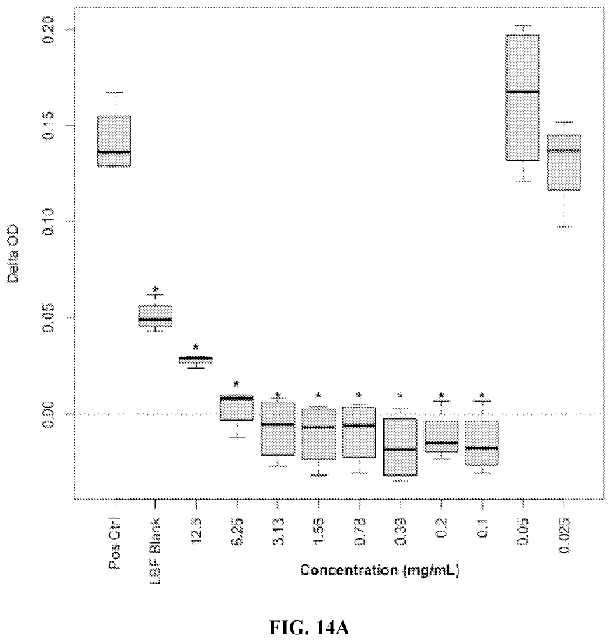

Results. As shown in FIG. 13A, there was inhibition of F. columnare when treated with PR-D9 at concentrations from 0.05-12.5 mg/mL, as seen in the diminished Delta OD₆₀₀ values post incubation relative to the positive control. The concentration of bacteria present in each well at the start of the liquid broth assay was 7×10⁶ CFU/mL. The OD₆₀₀ of positive controls increased from 0.023 to 0.25 through the 72 hr incubation. Significantly lower OD₆₀₀ values were observed ≥0.05 mg/mL PR-D9 compared with controls (FIG. 13A). Concentrations ≥0.78 mg/mL resulted in negative delta OD₆₀₀, suggesting some level of bacterial neutralization. However, the LBF control also had a negative delta OD₆₀₀, which was unexpected. Plating of 100 μL of the 12.5 mg/mL group on TYES media resulted in too many colonies to count, but did not create a full lawn of bacteria (FIG. 13B). As shown in FIG. 13C, the agar plate assay displayed inhibition of F. columnare by PR-D9.

Treatment with ≥3.13 mg/mL PR-D9 resulted in inhibition in all replicate plates, with inhibition areas ranging from 2-4 mm.

Accordingly, these results show that the Paenibacillus strain of the present technology, PR-D9, exhibits potent anti-F. columnare activity and is effective in methods for reducing or inhibiting the growth of F. columnare and in methods for preventing, reducing the risk of, or treating conditions associated with F. columnare including, but not limited to, columnaris disease.

### Example 14: The Paenibacillus Strain of the Present Technology Demonstrates Activity Against Renibacterium salmoninarum

This example demonstrates that the Paenibacillus strain of the present technology, PR-D9, exhibits potent anti-R. salmoninarum activity.

Methods. To test the efficacy of PR-D9 against R. salmoninarum growth in vitro, liquid broth and agar diffusion assays were carried out using a 1:2 dilution series of PR-D9 (as outlined in Table 4). Briefly, cryopreserved R. salmoninarum were streaked onto appropriate media and incubated (KDM2 for 29 days at 15° C.), then grown in broth (10 mL TYES for 14 days at 200 RPM at 15° C.). Equal volumes of R. salmoninarum and the appropriate PR-D9 dilution were mixed and an initial optical density point was taken. After 72 hours another reading was taken to measure changes in growth.

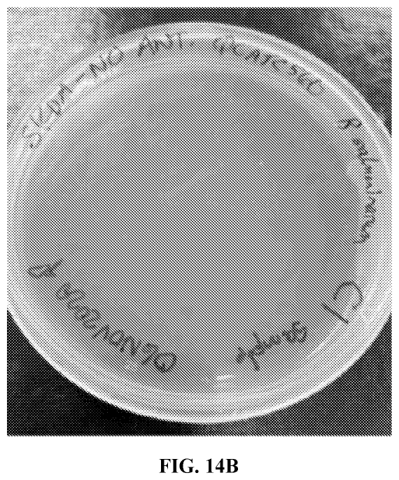

Results. As shown in FIG. 14A, there was inhibition of R. salmoninarum when treated with PR-D9 at concentrations from 0.1-12.5 mg/mL, as seen in the diminished Delta OD₆₀₀ values post incubation relative to the positive control. Initial concentration of R. salmoninarum was 3.0×10⁸. The OD₆₀₀ increased from 0.16 to 0.30 in controls through the 14-day incubation. The liquid broth assay displayed significant reductions in OD₆₀₀ values for concentrations of PR-D9 ranging from 12.5 mg/mL to 0.1 mg/mL when compared to positive controls. At these concentrations, no growth (delta OD₆₀₀=0) was observed. The LBF Blank sample (12.5 mg/mL LBF but no bacteria) and the 12.5 mg/mL treatment had slightly higher OD₆₀₀ values compared to concentrations between 6.25 and 0.10 (FIG. 14A) suggesting that growth from the PR-D9 was observed at 12.5 mg/mL. Plating of 100 μL from a combination of replicates from this group resulted in several small colonies (FIG. 14B). Concentrations of ≤0.05 mg/mL PR-D9 had no significant effect compared to control (FIG. 14A).

Accordingly, these results show that the Paenibacillus strain of the present technology, PR-D9, exhibits potent anti-R. salmoninarum activity and is effective in methods for reducing or inhibiting the growth of R. salmoninarum and in methods for preventing, reducing the risk of, or treating conditions associated with R. salmoninarum including, but not limited to, bacterial kidney disease (BKD).

### Example 15: The Paenibacillus Strain of the Present Technology Demonstrates Activity Against Piscrickettsia salmonis

This example demonstrates that the Paenibacillus strain of the present technology, PR-D9, exhibits potent anti-P. salmonis activity.

Methods. To test the efficacy of PR-D9 against P. salmonis growth, a qPCR plate assay was carried out using a dilution series of PR-D9. Briefly, a cell suspension of P. salmonis at 7.5×10⁶ TCID₅₀/mL was mixed with an equal volume of the appropriate PR-D9 dilution and incubated for 24 hours at 15° C. The plate assay included three dilutions of PR-D9 as shown in Table 5.

TABLE 5
Stock and diluted concentrations of PR-D9.

| Dilution | PR-D9 (mg/mL) |
|---|---|
| 1 | 12.50 |
| 2 | 0.78 |
| 3 | 0.10 |

Control wells were similarly incubated, including a positive control with P. salmonis alone, a PR-D9 control with no P. salmonis, and a media blank. DNA was then extracted from all samples and qPCR was performed to measure bacterial viability.

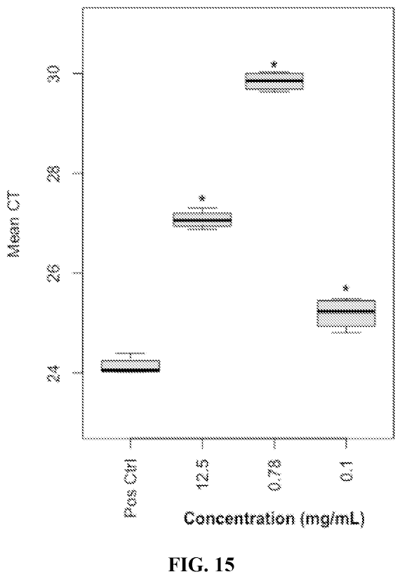

Results. As shown in FIG. 15, there was a decrease in P. salmonis viability when treated with PR-D9 at concentrations of 0.1, 0.78, and 12.5 mg/mL, as seen in the increases to CT values post incubation relative to the positive control. All three dilutions (Table 5) of PR-D9 had significantly higher CT values compared with positive controls. These difference however, were not intuitive, where 12.5 mg/mL resulted in ca. 3 CT reduction, 0.78 mg/mL caused ca. 5 CT reduction, and 0.1 mg/mL caused 1 CT reduction compared to control.

Accordingly, these results show that the Paenibacillus strain of the present technology, PR-D9, exhibits potent anti-P. salmonis activity and is effective in methods for reducing or inhibiting the growth of P. salmonis and in methods for preventing, reducing the risk of, or treating conditions associated with P. salmonis including, but not limited to, piscirickettiosis.

### Example 16: The Paenibacillus Strain of the Present Technology Demonstrates Activity Against White Spot Syndrome Virus (WSSV)

This example demonstrates that the Paenibacillus strain of the present technology, PR-D9, exhibits potent anti-WSSV activity.

Methods. To test the efficacy of PR-D9 against R. salmoninarum growth in vitro, liquid broth and agar diffusion assays were carried out using a 1:2 dilution series of PR-D9, as shown in Table 6.

TABLE 6
Stock and diluted concentrations of PR-D9.

| Dilution No. | PR-D9 (old)^{A} (mg/mL) | PR-D9 (new)^{A} (mg/mL) |
|---|---|---|
| 1 | 12.50 | 12.50 |
| 2 | 6.25 | 6.25 |
| 3 | 3.13 | 3.13 |
| 4 | 1.56 | 1.56 |
| 5 | 0.78 | 0.78 |
| 6 | 0.39 | 0.39 |
| 7 | 0.20 | N/Ap |
| 8 | 0.10 | N/Ap |
| 9 | 0.05 | N/Ap |
| 10 | 0.025 | N/Ap |

^{A}PR-D9 arrived in two shipments and thus are labeled old and new based on time of arrival.

Briefly, WSSV was grown in shrimp muscle that was then pulverized, homogenized (30 Hz for 30 seconds), centrifuged at 4° C. at 2000×g for 15 minutes, and finally filtered through a 0.45 μm filter to create a WSSV homogenate. Then 10 μl of the homogenate was mixed with 90 μl PBS and 100 μl of the appropriate PR-D9 dilution and incubated at 28° C. for 24 hours. DNA was then extracted and qPCR was performed to measure the number of viral particles.

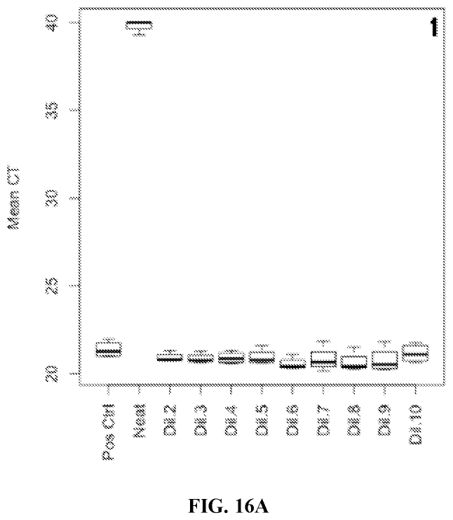

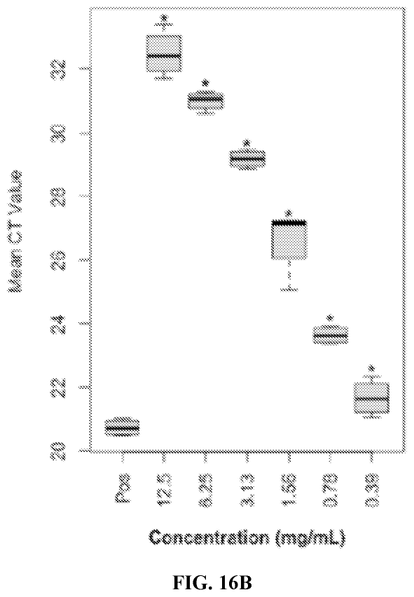

Results. As shown in FIG. 16A, the neat concentrations of PR-D9 showed significant decreases in viral particles compared to positive controls (i.e., significantly higher CT values). Only one of eight PR-D9 neat replicates (duplicate qPCR reactions on four wells) amplified, with a CT value of 38.582. As shown in FIG. 16B, there was a decrease in viable WSSV particles when treated with PR-D9 at concentrations from 0.39-12.5 mg/mL, as seen in the increases to CT values post incubation relative to the positive control.

Accordingly, these results show that the Paenibacillus strain of the present technology, PR-D9, exhibits potent anti-WSSV activity.

Summary of Results from Examples 2-16.

Three different assays were used in Examples 2-16: (i) the liquid broth assay; (ii) the agar diffusion assay, and (iii) the alternative plate assay with follow-up qPCR. In total, 12 bacteria and one oomycete were assayed using the liquid broth assay and agar diffusion assay. Another bacterium, P. salmonis, and White Spot Syndrome Virus (WSSV) were assayed with the alternative plate assay as growth could not be achieved in a cell-free environment.

Based on agar diffusion assays, PR-D9 inhibited growth in a broad range of pathogens at low concentrations. Growth inhibition was observed in 10 of 12 PR-D9-treated bacteria using agar diffusion assays, where A. salmonicida and S. agalactiae growth was not affected by treatment with PR-D9. In broth, PR-D9 inhibited all 12 bacteria tested, at minimum inhibitory concentrations (MIC) of 0.78-0.0025 mg/mL. The oomycete, S. parasitica was also inhibited by PR-D9 in broth (MIC=0.78 mg/mL), but not on agar.

Estimated MICs for PR-D9 were always lower in broth assays compared to agar assays (Table 7). This disparity may be due to differences in treatment exposure (i.e., dosing) between the two assay types, where broth assays had known concentrations of PR-D9 present in solution compared to unknown final concentrations in the agar diffusion assay.

TABLE 7
Minimum Inhibitory Concentrations (MIC; mg/mL) of PR-D9
on 15 aquatic pathogens. PR-D9 was used in broth and in agar
diffusion assays.

| | Minimum Inhibitory Concentration | |
|---|---|---|
| | (mg/mL) | |
| Pathogen | PR-D9 Broth Assay | PR-D9 Plate Assay |
| Whitespot Syndrome | N/Ap | 0.78^{B} |
| Virus | | |
| A. salmonicida | 0.39 | NE |
| F. psychrophilium | 0.10 | 1.56 |
| P. damselae piscida | 0.39 | 3.13 |
| V. anguillarum | 0.78 | 6.25 |
| V. parahaemolyticus | 0.050 | 6.25 |
| Y. ruckeri | 0.20 | 25.0 |
| S. agalactiae | 0.10 | 3.13 |
| S. iniae | 0.025 | 3.13 |
| P. salmonis | N/Ap | 0.10^{B} |
| S. parasitica | 0.78 | NE |
| T. maritimum | 0.025 | 12.5 |
| M. viscosa | 0.20 | 1.56 |
| F. columnare | 0.050 | 3.13 |
| R. salmoninarum | 0.10 | 3.13 |

^{B}Alternative plate assay quantified via qPCR
N/Ap = Not Applicable
NE = No effect

The relationship of PR-D9 concentration and patterns of pathogen growth inhibition in broth differed between pathogens treated with PR-D9, yielding three main patterns. The dose-response curve was observed for five pathogens including S. parasitica, F. columnare, A. salmonicida, F. psychrophilium, and Y. ruckeri.

A similar pattern of growth inhibition was observed for a further six bacteria; however, rather than a dose-response curve with a well-defined exponential phase, a maximal efficacy threshold was detected, where delta OD₆₀₀ was either the same or lower for concentrations between 12/5 mg/mL and the MIC. Concentrations of PR-D9 lower than this maximal efficacy threshold either inhibited growth negligibly (i.e., no significant difference from positive control) or showed slight growth inhibition (positive delta OD₆₀₀). Bacteria with PR-D9 efficacy thresholds included P. damselae, V. parahaemolyticus, V anguillarum, S. iniae, S. agalactiae, and R. salmoninarum.

The shape of the growth inhibition curve for T. maritimum, and M. viscosa did not fit either pattern. This difference may have resulted from adjustments made to PBS volumes in the assay to promote bacterial growth in positive samples for these assays, as 1:1 dilutions with pathogen and PR-D9 resulted in no growth of positive controls for these bacteria.

Two pathogens, WSSV and P. salmonis, were simply incubated with PR-D9 (i.e., no growth) prior to collection of material, DNA extraction, and qPCR targeting the pathogen. The purpose of the assays was to determine whether 24 hr incubations with PR-D9 caused a decrease in detectable copies of pathogen.

In summary, the lyophilized bacterial fermentation, PR-D9, was efficacious in inhibiting the growth or detectable copy number of all pathogens tested in broth, and 10 or 12 on agar. Plating of 12/5 mg/mL PR-D9 treated bacterial following the broth assay resulted in growth of PR-D9 alone, indicating the treatment killed the pathogens and did not merely inhibit growth. Accordingly, these results demonstrate that the Paenibacillus strain, PR-D9, of the present technology is useful in methods for inhibiting the growth of aquaculture pathogen growth and in methods for reducing the risk of, preventing, or treating an aquaculture pathogen infection.

### Example 17: The Paenibacillus Strain of the Present Technology Demonstrates In Vivo Activity Against Vibrio parahaemolyticus in a Whiteleg Shrimp Model of Acute Hepatopancreatic Necrosis Disease (AHPND)

This example demonstrates that the Paenibacillus strain, PR-D9, of the present technology is effective in methods for reducing the risk of, preventing, or treating acute hepatopancreatic necrosis disease (AHPND), which is caused by infection with Vibrio parahaemolyticus (VpAHPND). To test the efficacy of PR-D9 against V. parahaemolyticus in vivo, a challenge assay using whiteleg shrimp (Litopenaeus vannamei) was performed.

### Methods

Test article and test system. Briefly, 500 juvenile whiteleg shrimp of mean weight 3.08 g (SD 0.29) were pre-fed one of a number of diets including an unsupplemented control feed (“Feed A”; “Treatment A”; 0% inclusion of PR-D9) or feed supplemented with 1% (“Feed B”; “Treatment B”; 1% inclusion of PR-D9), 5% (“Feed C”; “Treatment C”; 5% inclusion of PR-D9), or 10% (“Feed D”; “Treatment D”; 10% inclusion of PR-D9) w/w PR-D9, for 22 days prior to challenge. As a positive control a fifth group was given an unsupplemented feed with an added antibiotic treatment 10 days prior to challenge (“Feed E”; “Treatment E”). The feed formulation for the treatment feeds are shown in Table 8.

TABLE 8
Formulation table for the study feeds.

| | Inclusion (% as-fed) | | | |
|---|---|---|---|---|
| | Feed A | Feed B | Feed C | Feed D |
| Ingredients | (Control) | (1% PR-D9) | (5% PR-D9) | (10% PR-D9) |
| Blood meal | 5.000 | 4.950 | 4.750 | 4.500 |
| (Spray-dried) | | | | |
| Carophyll Pink | 0.050 | 0.050 | 0.048 | 0.045 |
| CGM | 11.328 | 11.215 | 10.762 | 10.195 |
| Cholesterol | 0.190 | 0.188 | 0.181 | 0.171 |
| DL Methionine | 0.106 | 0.105 | 0.101 | 0.095 |
| Fish oil herring | 6.768 | 6.700 | 6.430 | 6.091 |
| FM Herring | 15.000 | 14.850 | 14.250 | 13.500 |
| L-Lysine | 0.667 | 0.660 | 0.634 | 0.600 |
| L-Threonine | 0.191 | 0.189 | 0.181 | 0.172 |
| MCP 21% P | 2.985 | 2.955 | 2.836 | 2.687 |
| PR-D9 | 0.000 | 1.000 | 5.000 | 10.000 |
| SBM | 30.000 | 29.700 | 28.500 | 27.000 |
| (solvent-extracted) | | | | |
| Soy lecithin | 1.200 | 1.188 | 1.140 | 1.080 |
| Stay-C | 0.500 | 0.495 | 0.475 | 0.450 |
| Vit & Min Premix | 0.500 | 0.495 | 0.475 | 0.450 |
| WGM | 2.500 | 2.475 | 2.375 | 2.250 |
| Wheat flour | 23.015 | 22.785 | 21.864 | 20.714 |
| Total | 100.000 | 100.000 | 100.000 | 100.000 |

Shrimp distribution into tanks. The study utilized five treatments, which were designated as A-E in sequence (see Table 9). Each treatment was administered to four replicate tanks, with each tank of 40 L capacity receiving 25 shrimp. Shrimp were bulk weighed upon distribution.

TABLE 9
Treatment group information.

| | Treatment Feed | | Number of |
|---|---|---|---|
| Treatment | with Enrichment | Inclusion Rate | replicate tanks |
| A | A | 0% (Control) | 4 |
| B | B | 1% | 4 |
| C | C | 5% | 4 |
| D | D | 10% | 4 |
| E | E (Doxycycline) | 100 mg/kg feed | 4 |

Pre-challenge feeding. Shrimp were fed with ca. 10% ration/day determined based on initial tank biomass and provided in 3 hand feeding during the day. Shrimp in treatment E received antibiotic (Doxycycline @100 mg/Kg feed) supplemented control feed from 10 days prior to challenge at same ration as previous. Treatment E feed was prepared by mixing the antibiotic to control feed followed by 1% (v/w) canola oil top coating. The prepared feed was stored at 4° C. Study feeds were administered for 22 days; shrimp were bulk weighed on 9 Feb. 2021. Bulk weight data was used to calculate challenge feed amount to be dispensed in each tank, and analyze weight gain, treatment effect.

Tank distribution for challenge. The number of shrimp remaining in each tank was counted at the end of feeding phase. After all tank populations were counted each was equalized to 21 shrimp to ensure equal initial challenge populations (see Table 10).

TABLE 10
Number of shrimp housed in each tank, recovered post feeding,
culled, and challenged during the study.

| | | | Population | | |
|---|---|---|---|---|---|
| | | | Remining | | |
| | | Initial | Post | | |
| | | Population | Pre-Challenge | Number | Number |
| Treatment | Tank | at the start | Feeding | Culled | Challenged |
| A | H1 | 25 | 23 | 2 | 21 |
| | H5 | 25 | 22 | 3 | 21 |
| | H6 | 25 | 25 | 4 | 21 |
| | H8 | 25 | 23 | 2 | 21 |
| B | H4 | 25 | 25 | 4 | 21 |
| | H7 | 25 | 21 | 0 | 21 |
| | H9 | 25 | 23 | 2 | 21 |
| | H16 | 25 | 25 | 4 | 21 |
| C | H11 | 25 | 21 | 0 | 21 |
| | H15 | 25 | 24 | 3 | 21 |
| | H18 | 25 | 23 | 2 | 21 |
| | H19 | 25 | 22 | 1 | 21 |
| D | H3 | 25 | 23 | 2 | 21 |
| | H14 | 25 | 23 | 2 | 21 |
| | H17 | 25 | 22 | 1 | 21 |
| | H20 | 25 | 23 | 2 | 21 |
| E | H2 | 25 | 21 | 0 | 21 |
| | H10 | 25 | 23 | 2 | 21 |
| | H12 | 25 | 23 | 2 | 21 |
| | H13 | 25 | 24 | 3 | 21 |

Disease challenge. Typical feeding regimen was not carried out on the day of challenge, 9 Feb. 2021. Instead, shrimp received a single feeding of Zeigler Raceway at 5% body weight (equalized across all tanks) which was top coated with V. parahaemolyticus culture. Treatment diets resumed the next day, but feeding ration was reduced to ca. 3-5% due to decreased appetite and diminishing numbers resulting from mortalities. Treatment E tanks continued to receive antibiotic supplemented feed for a further four days before returning to control diet. Feeding continued until termination on 19 Feb. 2021.

Daily husbandry and necropsy. Physico-chemical and water quality parameters were maintained within the defined range of the study protocol without any deviation during the study period. Shrimp were monitored twice daily for husbandry and welfare checks. Any mortality and signs of morbidity with presence or absence of disease symptoms were thoroughly necropsied to record gross external and internal pathology. In addition, hepatopancreas was collected from 10 mort/treatment fresh infected mortality/moribund post challenge for further investigations, if required. Similarly, hepatopancreas sample was collected from survivors at termination, 10 mort/treatment. Hepatopancreas was preserved in 1 ml RNAlater and stored at 4° C. for 24-48 hours before long term storage at −20° C.

Statistical analysis. Pre-challenge bulk weight, and weight gain post feeding were analyzed by Analysis of Variance (ANOVA) where test assumptions were met by the residuals passing normality tests. Tukey's multiple comparisons test was done where significant differences were observed. Survival analysis post challenge were analyzed by Long-Rank (Mantel-Cox) test. Statistical analyses were done in GraphPad Prism software (9.0.2).

### Results

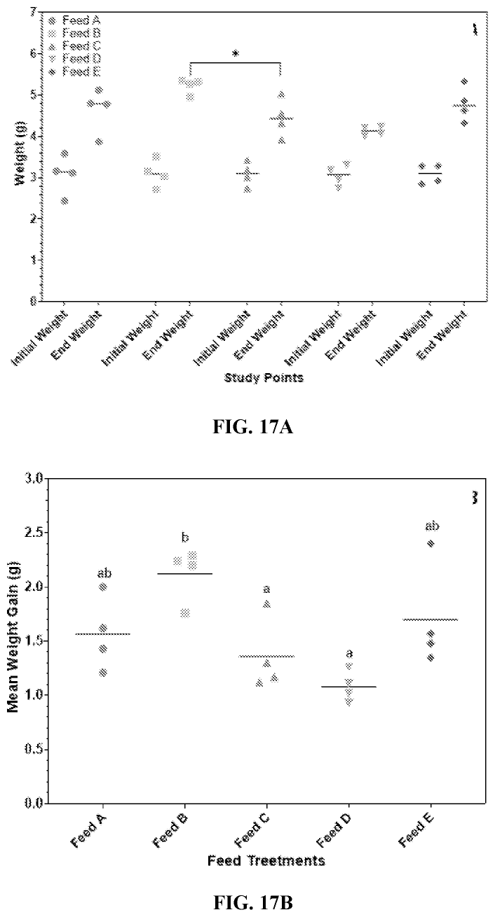

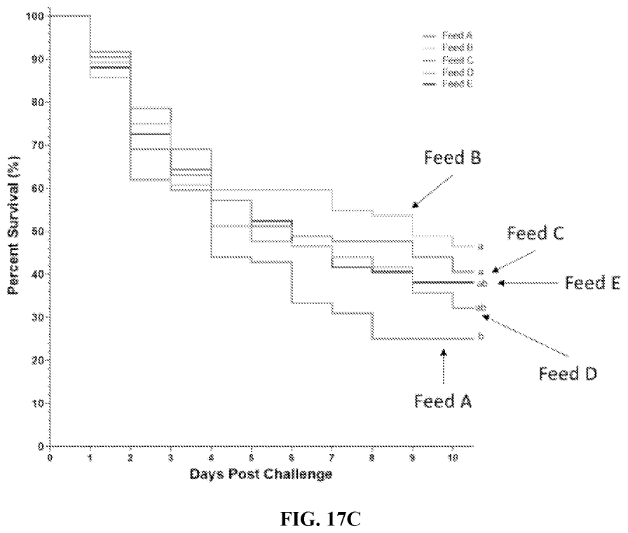

Pre-challenge growth. Pre-challenge growth was calculated via tank bulk weight assessments, at initial distribution and after 22 days of feeding. FIGS. 17A and 17B illustrate start and end weights of tanks which received each treatment (FIG. 17A) and then the calculated mean growth per shrimp (FIG. 17B). In brief, tanks which received treatment B (PR-D9 at 1% w/w) had significantly greater growth than that which received treatments C, p=0.03 (PR-D9 at 5% w/w) and D, p=0.003 (PR-D9 at 10% w/w), but not treatment A or E both of which had 0% PR-D9 enrichment.

Post-challenge mortality. A significant treatment effect on shrimp survival was observed in this study. PR-D9, when utilized at 1% and 5%, significantly increased survival compared to control (21% more survival in B than A, p=0.04; 15% more survival in C than A, p=0.03; Table 11).

TABLE 11
Actual percent mortality and survival, percent higher
survival compared to control, and relative percent
survival of shrimp in different treatments fed with
different treatment feeds post challenge (V. parahaemolyticus)
over the duration of days.

| | | | % Higher | Relative |
|---|---|---|---|---|
| | | | Survival | Percent |
| | Actual % | Actual % | Compared to | Survival |
| Treatments | Mort | Survival | Control* | (RPS) ** |
| A (0% PR- | 75% | 25% | 0% | 0% |
| D9/Control) | | | | |
| B (1% PR-D9) | 54% | 46% | 21% | 28% |
| C (5% PR-D9) | 60% | 40% | 15% | 20% |
| D (10% PR-D9) | 68% | 32% | 7% | 9.33% |
| E (Antibiotic) | 62% | 38% | 13% | 17.33% |

*Calculated as % Higher Survival = % Survival of the Treatment − %
Survival of the Control
**Calculated as Relative Percent Survival =
$( 1 - \frac{\% Mortality of the Treatment}{\% Mortality of the Control} ) \times 100 ;$ according to Amend 1981.

FIG. 17C shows survival curves according to treatment, with annotation of significantly different results. The study demonstrates a strong effect of PR-D9, but in a dose dependent manner, as when 10% inclusion was used survival was not significantly greater compared to that of the control group. Without wishing to be bound by theory, acute mortalities in the experiment are most likely caused by V. parahaemolyticus produced binary toxins PirAvp and PirBvp, i.e., toxins already present in culture media then ingested by shrimp at challenge. After initial mortality due toxins, ingested bacteria colonize the gut causing further mortalities with clinical signs such as pale discoloration and atrophy of hepatopancreas, empty stomach and mid gut. Without wishing to be bound by theory, given the proposed mode of action of PR-D9 it is hypothesized that treatments B and C had greater survival compared to control group as the enrichment reduced colonization of the Gut by the Bacteria.

### CONCLUSION

The present study clearly demonstrated that lower inclusion level of PR-D9 (1% w/w) in the diet has significant effect on growth, and providing protection against AHPND in whiteleg shrimp. Despite relatively lower growth, 5% PR-D9 provided similar protection against the disease compared to 1% inclusion level. These results demonstrate that PR-D9 has an impact on survival during AHPND. Accordingly, these results show that the Paenibacillus strain of the present technology, PR-D9, is useful as a feed additive in methods for increasing the growth of animals, in methods for reducing the risk of, preventing, or treating AHPND, and in methods for increasing survival in animals suffering from APHND.

### Example 18: Methanol Extract Assay to Assess Bio-Activity of Extracellular and Intracellular Compounds of the Paenibacillus Strain of the Present Technology

Methods. To test the anti-pathogenic potential of both extracellular and intracellular compounds, a methanol extract assay was performed. Briefly, a fermentation of strain PR-D9 was obtained, and 10 mL was removed containing both cells and broth. 10 mL of 100% MeOH was added to yield a 50% methanol concentration, the tube was vortexed to break up cells and make homogenous. Then, 5 μL of the extract was spotted onto a Mueller Hinton Agar+2% NaCl agar plate which had been overlaid with a culture of Vibrio parahaemolyticus strain A3 at an OD of 0.005. The 50% methanol extract was also diluted 1:2 in a serial fashion in 50% Methanol in order to find the dilution point at which bio-activity becomes extinct.

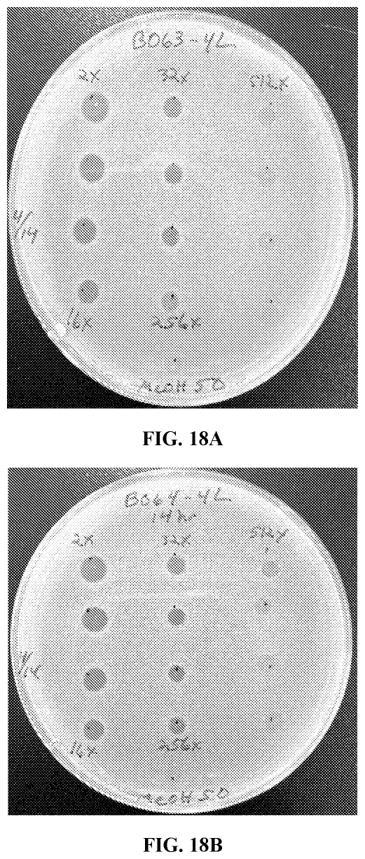

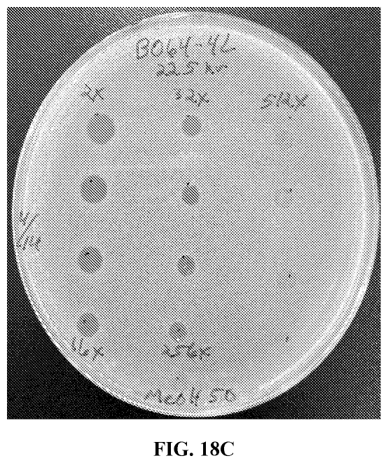

Results. As shown in FIGS. 18A-C, anti-pathogenic activity was observed at most dilutions of the methanol extract, starting to diminish between the 1/256-1/512 dilution points.

Accordingly, these results demonstrate that both extracellular and intracellular compounds of the Paenibacillus strain of the present technology are useful in methods for reducing the risk of, or preventing, or treating an aquaculture pathogen infection.

### Example 19: The Paenibacillus Strain of the Present Technology Retains Anti-Pathogenic Activity When Applied With Excipients

This example demonstrates that the Paenibacillus strain of the present technology, PR-D9, retains its anti-pathogenic activity when applied with excipients (e.g., lyophilization protectants).

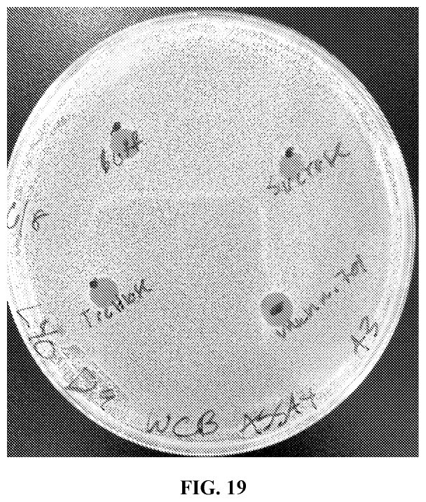

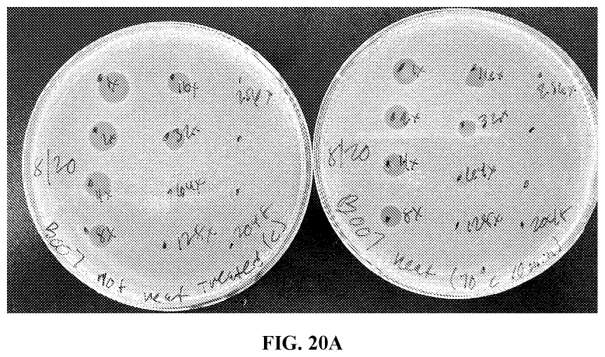

To test the effect of excipients on the anti-pathogenic activity of PR-D9, a zone of clearance assay was carried out with four excipients. Briefly, a spot of resuspended, lyophilized PR-D9 (5 μL) mixed with one of sucrose, mannitol, trehalose, or OPS buffer was applied to a plate of Vibrio parahaemolyticus. As shown in FIG. 19, there was a zone of clearance of V. parahaemolyticus around each treatment.

Accordingly, these results show that the addition of an excipient to the Paenibacillus strain of the present technology, PR-D9, did not impact the anti-pathogenic activity.

### Example 20: The Paenibacillus Strain of the Present Technology Retains Anti-Pathogenic Activity Despite Heat Treatment and Autoclaving

This example demonstrates that the Paenibacillus strain of the present technology, PR-D9, exhibits potent anti-pathogenic activity despite heat treatment.

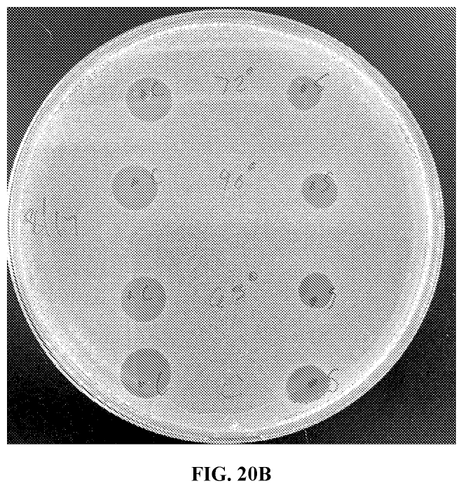

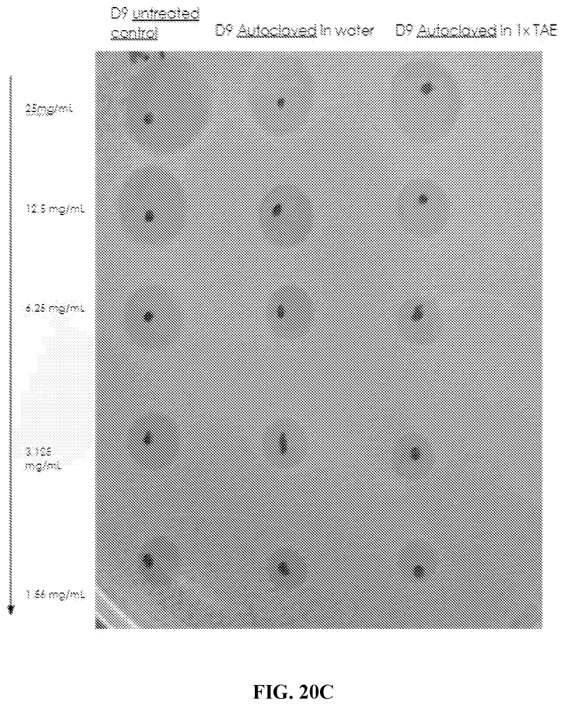

To test the heat susceptibility of PR-D9's anti-pathogenic activity, a series of heat exposure experiments were carried out. First, 100 μl of whole PR-D9 fermentation broth was incubated at 70° C. for 10 minutes before being spotted onto a MHA+3% NaCl plate of V. parahaemolyticus in a serial dilution. As shown in FIG. 20A, heat treatment did not abrogate the anti-pathogenic activity of PR-D9 broth, as indicated by the zones of clearance around the heated sample treatment spots. Second, PR-D9 supernatant and cells (pelleted and resuspended in PBS) were exposed to one of three heat conditions: 72° C. for 15 seconds, 90° C. for 5 minutes, or 63° C. for 30 minutes. The cells and the supernatant, along with untreated controls, were then spotted onto a MHA+3% NaCl plate of V. parahaemolyticus. As shown in FIG. 20B, heat treatment did not abrogate the anti-pathogenic activity of either the PR-D9 cells or the growth supernatant, as indicated by the zones of clearance around the heated sample treatment spots. Finally, lyophilized PR-D9 powder was resuspended in water or 1×TAE buffer at various concentrations (25, 12.5, 6.25, 3.125, or 1.56 mg/mL) and then autoclaved at 121° C. and 15 PSI for 15 minutes. The autoclaved resuspensions, and an un-autoclaved control, were spotted onto a MHA+3% NaCl plate covered with V. parahaemolyticus. As shown in FIG. 20C, autoclaving did not abrogate the anti-pathogenic activity of lyophilized PR-D9 at any concentration in either resuspension, as indicated by the zones of clearance around the autoclaved sample treatment spots.

Accordingly, these results show that the Paenibacillus strain of the present technology, PR-D9, retains anti-pathogenic activity despite exposure to a range of high heat conditions, including the intense heat and pressure of an autoclave.

### Example 21: The Paenibacillus Strain of the Present Technology Retains Anti-Pathogenic Activity After Spray Drying

This example demonstrates that the Paenibacillus strain of the present technology, PR-D9, retains potent anti-pathogenic activity after being spray dried.

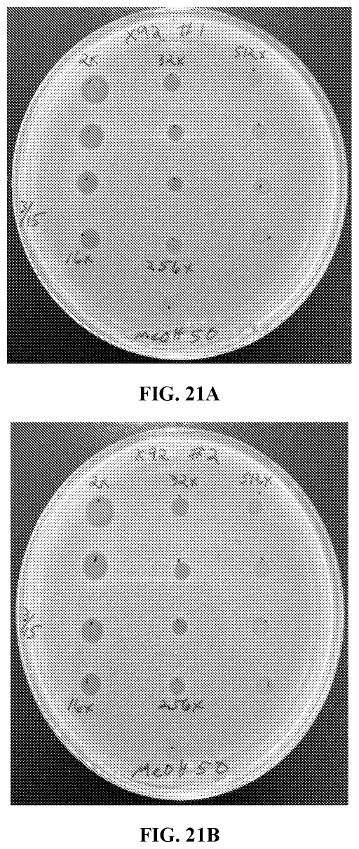

To test the impact of spray drying on PR-D9's anti-pathogenic activity, a zone of clearance assay was carried out. Briefly, entire fermentation of PR-D9 was spray dried under 170° C. inlet and 90° C. outlet temperatures. Spray dried samples were resuspended 1:2 in 50% methanol, serially diluted 1:2 in 50% methanol, and spotted onto a MHA+3% NaCl plate covered in Vibrio parahaemolyticus. As shown in FIGS. 21A and 21B, there was a zone of clearance of V. parahaemolyticus around each treatment spot down to a dilution of 1/256-512x.

Accordingly, these results show that the Paenibacillus strain of the present technology, PR-D9, retains anti-pathogenic activity after a spray drying procedure, and is useful in methods for reducing the risk of, or preventing, or treating an aquaculture pathogen infection.

### Example 22: The Paenibacillus Strain of the Present Technology Secretes Soy Nutrient Digesting Enzymes

This example demonstrates that the Paenibacillus strain of the present technology, PR-D9, secretes enzymes that digest soy nutrients.

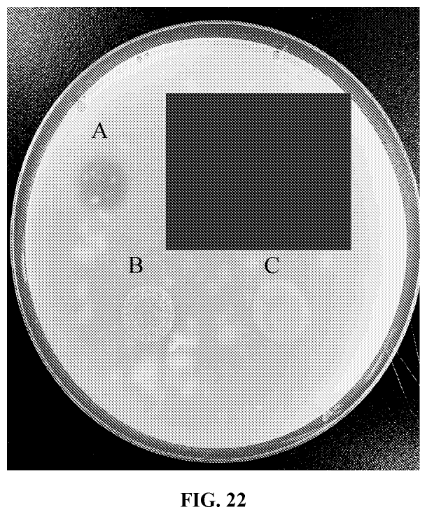

To test the ability of PR-D9 to digest extracellular soy nutrients, a soy digestion assay was carried out. Briefly, 5 μL each of PR-D9 fermentation and two commonly used industry probiotics, Biomin and Pro-2, were spotted onto a soy-flower mannitol plate and incubated at 30° C. for 24 hours. As shown in FIG. 22, there was a zone of clearance around the PR-D9 application (A), while the other probiotics (B, C) had no such effect.

Accordingly, these results show that the Paenibacillus strain of the present technology, PR-D9, secretes exoenzymes that are able to digest extracellular soy nutrients, and demonstrate that the strain is useful in methods for improving the nutritional availability of soy protein to an aquaculture animal.

### Example 23: The Paenibacillus Strain of the Present Technology Demonstrates In Vivo Activity Against Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolenia parasitica, Tenacibaculum maritimum, Moritella viscosa, Flavobacterium columnare, and Renibacterium salmoninarum in Aquaculture Models

This example prophetically demonstrates that the Paenibacillus strain, PR-D9, of the present technology is effective in methods for reducing the risk of, preventing, or treating aquaculture pathogen infections and the diseases that they cause. To test the efficacy of PR-D9 against Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum in vivo, challenge assays using any acceptable animal model, such as, but not limited to, the aquaculture animals described herein, are performed.

### Methods

The illustrative method described below is not intended to be limiting, and is based on the experimental procedure of Example 17.

Test article and test system. Briefly, aquaculture animals are pre-fed one of a number of diets, such as those described in Example 17 (e.g., an unsupplemented control feed (“Feed A”; “Treatment A”; 0% inclusion of PR-D9) or feed supplemented with 1% (“Feed B”; “Treatment B”; 1% inclusion of PR-D9), 5% (“Feed C”; “Treatment C”; 5% inclusion of PR-D9), or 10% (“Feed D”; “Treatment D”; 10% inclusion of PR-D9) w/w PR-D9), for approximately 22 days prior to challenge. As a positive control a fifth group is given an unsupplemented feed with an added antibiotic treatment about 10 days prior to challenge (“Feed E”; “Treatment E”). An illustrative feed formulation for the treatment feeds is provided above in Table 8.

Aquaculture animal distribution into tanks. The study utilizes five treatments, which are designated as A-E in sequence (see, e.g., Table 9 above). Each treatment is administered to four replicate tanks. Animals are observed for general health characteristics upon distribution.

Pre-challenge feeding. Animals are fed with ca. 10% ration/day determined based on initial tank biomass and provided in 3 hand feeding during the day. Study feeds are administered for about 22 days.

Disease challenge. Typical feeding regimen is not carried out on the day of challenge. Instead, the aquaculture animals receive a single feeding which is top coated with Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, or Renibacterium salmoninarum culture. Treatment diets resume the next day.

Daily husbandry and necropsy. Physico-chemical and water quality parameters are maintained within the defined range of the study protocol without any deviation during the study period. Animals are monitored twice daily for husbandry and welfare checks. Any mortality and signs of morbidity with presence or absence of disease symptoms are thoroughly necropsied to record gross external and internal pathology.

### Results

It is anticipated that administration of compositions comprising PR-D9 in the diet of the aquaculture animals will: reduce the risk of, or prevent, or treat an aquaculture pathogen infection: increase the survival of an aquaculture animal exposed to an aquaculture pathogen, and/or improve the growth of an aquaculture animal. Accordingly, these results will show that the Paenibacillus strain of the present technology, PR-D9, is useful as a feed additive in methods for increasing the growth of animals, in methods for reducing the risk of, preventing, or treating aquaculture pathogen infection, and in methods for increasing survival in animals suffering from an aquaculture pathogen infection.

### Biological Deposits

The Applicant requests that a sample of the deposited microorganism should be made available only to an expert approved by the Applicant.

Paenibacillus species strain PR-D9 deposited with the Agricultural Research Culture Collection (NRRL) International Depositary Authority, 1815 N. University Street, Peoria, Illinois 61604 U.S.A., on 28 Apr. 2021, under NRRL Accession Number B-68028.

The deposit was made according to the Budapest treaty on the international recognition of the deposit of microorganisms for the purposes of patent procedure.

What is claimed is:

1. An isolated Paenibacillus strain PR-D9 deposited with the Agricultural Research Culture Collection (NRRL—Northern Regional Research Laboratory) under NRRL Accession Number B-68028 or a variant strain thereof.

2. The isolated Paenibacillus strain of claim 1, wherein the Paenibacillus strain is a lyophilized bacterial fermentation (LBF).

3. The isolated Paenibacillus strain of claim 1, wherein the Paenibacillus strain is spray dried.

4. The isolated Paenibacillus strain of any one of claims 1-3, wherein the Paenibacillus strain is heat-stable.

5. The isolated Paenibacillus strain of any one of claims 1-4, wherein the Paenibacillus strain is capable of reducing or inhibiting growth of one or more pathogens selected from the group consisting of Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum, or any combination thereof.

6. The isolated Paenibacillus strain of claim 5, wherein the Paenibacillus strain is capable of inhibiting growth of one or more pathogens selected from the group consisting of Vibrio parahaemolyticus, Aeromonas salmonicida, Flavobacterium psychrophilium, Photobacterium damselae piscida, Vibrio anguillarum, Yersinia ruckeri, Streptococcus agalactiae, Streptococcus iniae, Saprolegnia parasitica, Tenacibaculum maritimum, Moritella viscosa, Flavobacterium columnare, and Renibacterium salmoninarum, at minimum inhibitory concentrations (MIC) of 0.78-0.025 mg/mL.

7. The isolated Paenibacillus strain of claim 5 or claim 6, wherein the Paenibacillus strain, after having been subjected to a heat treatment to form a heat-treated Paenibacillus strain, is capable of reducing or inhibiting the growth of the one or more pathogens.

8. The isolated Paenibacillus strain of claim 7, wherein the heat-treated Paenibacillus strain is capable of reducing or inhibiting pathogen growth to an extent that is substantially the same as a control isolated Paenibacillus strain PR-D9 that was not subjected to heat treatment.

9. The isolated Paenibacillus strain of claim 8, wherein the pathogen is Vibrio parahaemolyticus.

10. The isolated Paenibacillus strain of any one of claims 7-9, wherein the heat treatment comprises autoclaving.

11. The isolated Paenibacillus strain of claim 10, wherein the autoclaving is performed at a temperature of 121° C. and pressure of 15 psi for 15 minutes.

12. The isolated Paenibacillus strain of any one of claims 1-11, wherein, when the strain is fed to an aquaculture animal, the strain reduces the risk of, prevents, or treats one or more conditions selected from the group consisting of acute hepatopancreatic necrosis disease (AHPND), early mortality syndrome (EMS), white feces disease (WFD), white spot syndrome, piscirickettiosis, photobacteriosis, meningitis, bacterial kidney disease (BKD), tenacibaculosis, white ulcer disease, columnaris disease, furunculosis, vibriosis, and bacterial cold water disease (BCWD) in the aquaculture animal as compared to control aquaculture animal.

13. The isolated Paenibacillus strain of any one of claims 1-12, wherein, when the strain is fed to an aquaculture animal, the strain increases one or more of the growth and survival of the aquaculture animal as compared to a control aquaculture animal.

14. The isolated Paenibacillus strain of claim 13, wherein the aquaculture animal is a crustacean or cultured fish.

15. The isolated Paenibacillus strain of claim 14, wherein the aquaculture animal is a crustacean.

16. The isolated Paenibacillus strain of claim 15, wherein the crustacean is shrimp.

17. The isolated Paenibacillus strain of any one of claims 1-16, wherein the Paenibacillus strain produces exoenzymes that digest soy protein.

18. A food or feed additive comprising the isolated Paenibacillus strain of any one of claims 1-17.

19. The food or feed additive of claim 18, wherein the food is an aquaculture food and the feed additive is an aquaculture feed additive.

20. The aquaculture food of claim 19, wherein the aquaculture food comprises 0.25%, 0.5%, 1%, 5%, or 10% (w/w) Paenibacillus strain.

21. The aquaculture food of claim 20, wherein the Paenibacillus strain is a lyophilized bacterial fermentation.

22. A composition comprising:
(i) an isolated Paenibacillus strain PR-D9 deposited with the Agricultural Research Culture Collection (NRRL—Northern Regional Research Laboratory) under NRRL Accession Number B-68028 or a variant strain thereof; and
(ii) an excipient.

23. The composition of claim 22, wherein the Paenibacillus strain is lyophilized.

24. The composition of claim 22 or claim 23, wherein the Paenibacillus strain is a lyophilized bacterial fermentation (LBF).

25. The composition of claim 22, wherein the Paenibacillus strain is spray dried.

26. The composition of any one of claims 22-25, wherein the excipient comprises one or more lyoprotectants selected from the group consisting of mannitol, trehalose, sucrose, glycerol, glycine, skim milk, bovine serum albumin (BSA), and a lyophilization buffer, or any combination thereof.

27. The composition of any one of claims 22-26, wherein the Paenibacillus strain is heat-stable.

28. The composition of any one of claims 22-27, wherein the composition is capable of reducing or inhibiting growth of one or more pathogens selected from the group consisting of Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum, or any combination thereof.

29. The composition of claim 28, wherein the composition is capable of inhibiting growth of one or more pathogens selected from the group consisting of Vibrio parahaemolyticus, Aeromonas salmonicida, Flavobacterium psychrophilium, Photobacterium damselae piscida, Vibrio anguillarum, Yersinia ruckeri, Streptococcus agalactiae, Streptococcus iniae, Saprolegnia parasitica, Tenacibaculum maritimum, Moritella viscosa, Flavobacterium columnare, and Renibacterium salmoninarum, at minimum inhibitory concentrations (MIC) of 0.78-0.025 mg/mL.

30. The composition of any one of claims 22-29, wherein the composition, after having been subjected to a heat treatment to form a heat-treated composition, is capable of reducing or inhibiting the growth of the one or more pathogens.

31. The composition of claim 30, wherein the heat-treated composition is capable of reducing or inhibiting pathogen growth to an extent that is substantially the same as a control composition that was not subjected to heat treatment.

32. The composition of claim 30 or claim 31, wherein the heat treatment comprises autoclaving.

33. The composition of claim 32, wherein the autoclaving is performed at a temperature of 121° C. and pressure of 15 psi for 15 minutes.

34. The composition of any one of claims 22-33, wherein, when the composition is fed to an aquaculture animal, the strain reduces the risk of, prevents, or treats one or more conditions selected from the group consisting of acute hepatopancreatic necrosis disease (AHPND), early mortality syndrome (EMS), white feces disease (WFD), white spot syndrome, piscirickettiosis, photobacteriosis, meningitis, bacterial kidney disease (BKD), tenacibaculosis, white ulcer disease, columnaris disease, furunculosis, vibriosis, and bacterial cold water disease (BCWD) in the aquaculture animal.

35. The composition of any one of claims 22-34, wherein, when the composition is fed to an aquaculture animal, the composition increases one or more of the growth and survival of the aquaculture animal as compared to a control aquaculture animal.

36. The composition of claim 35, wherein the aquaculture animal is a crustacean or cultured fish.

37. The composition of claim 36, wherein the aquaculture animal is a crustacean.

38. The composition of claim 37, wherein the crustacean is shrimp.

39. The composition of any one of claims 22-38, wherein the composition is formulated for oral administration.

40. The composition of any one of claims 22-39, wherein the Paenibacillus strain produces exoenzymes that digest soy protein.

41. The composition of any one of claims 22-40, wherein the composition is formulated as food or a feed additive.

42. The composition of any one of claims 22-40, wherein the composition is formulated as an aquaculture food or an aquaculture feed additive.

43. The composition of claim 42, wherein the composition is formulated as an aquaculture food comprising 1%, 5%, or 10% (w/w) Paenibacillus strain.

44. The composition of claim 43, wherein the Paenibacillus strain is a lyophilized bacterial fermentation.

45. The composition of any one of claims 22-44, wherein the composition is formulated as an aquaculture bath.

46. A method of reducing the risk of, or preventing, or treating an aquaculture pathogen infection in a subject in need thereof, comprising administering to the subject an isolated Paenibacillus strain PR-D9 deposited with the Agricultural Research Culture Collection (NRRL—Northern Regional Research Laboratory) under NRRL Accession Number B-68028 or a variant strain thereof.

47. The method of claim 46, wherein the Paenibacillus strain is formulated as a composition, wherein the composition comprises: (i) the Paenibacillus strain; and (ii) an excipient.

48. The method of claim 46 or claim 47, wherein the Paenibacillus strain is lyophilized.

49. The method of claim 48, wherein the lyophilized Paenibacillus strain is a lyophilized bacterial fermentation (LBF).

50. The method of claim 46 or claim 47, wherein the Paenibacillus strain is spray dried.

51. The method of any one of claims 47-50, wherein the excipient comprises one or more lyoprotectants selected from the group consisting of mannitol, trehalose, sucrose, glycerol, glycine, skim milk, bovine serum albumin (BSA), and a lyophilization buffer, or any combination thereof.

52. The method of any one of claims 46-51, wherein the Paenibacillus strain is heat-stable.

53. The method of any one of claims 46-52, wherein reducing the risk of, or preventing, or treating an aquaculture pathogen infection in an aquaculture environment comprises reducing or inhibiting growth of one or more pathogens selected from the group consisting of Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum, or any combination thereof.

54. The method of any one of claims 46-53, wherein the aquaculture pathogen infection comprises one or more conditions selected from the group consisting of acute hepatopancreatic necrosis disease (AHPND), early mortality syndrome (EMS), white feces disease (WFD), white spot syndrome, piscirickettiosis, photobacteriosis, meningitis, bacterial kidney disease (BKD), tenacibaculosis, white ulcer disease, columnaris disease, furunculosis, vibriosis, and bacterial cold water disease (BCWD).

55. The method of any one of claims 46-54, wherein the subject is an aquaculture animal.

56. The method of claim 55, wherein the aquaculture animal is a crustacean or cultured fish.

57. The method of claim 56, wherein the aquaculture animal is a crustacean.

58. The method of claim 57, wherein the crustacean is shrimp.

59. The method of claim 57, wherein the pathogen is Vibrio parahaemolyticus.

60. The method of claim 58 or claim 59, wherein the aquaculture pathogen infection is acute hepatopancreatic necrosis disease (AHPND).

61. The method of any one of claims 46-60, wherein the Paenibacillus strain is administered orally to the subject.

62. The method of any one of claims 46-61, wherein the Paenibacillus strain is administered to the subject as food or a feed additive.

63. The method of any one of claims 46-61, wherein the Paenibacillus strain is administered to the subject as an aquaculture food or an aquaculture feed additive.

64. The method of claim 63, wherein the aquaculture food comprises 1%, 5%, or 10% (w/w) Paenibacillus strain.

65. The method of claim 64, wherein the Paenibacillus strain is a lyophilized bacterial fermentation.

66. A method for increasing the survival of an aquaculture animal exposed to an aquaculture pathogen, comprising administering to the subject an isolated Paenibacillus strain PR-D9 deposited with the Agricultural Research Culture Collection (NRRL—Northern Regional Research Laboratory) under NRRL Accession Number B-68028 or a variant strain thereof, wherein the survival of the aquaculture animal is increased as compared to an untreated control.

67. The method of claim 66, wherein the Paenibacillus strain is formulated as a composition, wherein the composition comprises: (i) the Paenibacillus strain; and (ii) an excipient.

68. The method of claim 66 or claim 67, wherein the Paenibacillus strain is lyophilized.

69. The method of claim 68, wherein the lyophilized Paenibacillus strain is a lyophilized bacterial fermentation (LBF).

70. The method of claim 66 or claim 67, wherein the Paenibacillus strain is spray dried.

71. The method of any one of claims 67-70, wherein the excipient comprises one or more lyoprotectants selected from the group consisting of mannitol, trehalose, sucrose, glycerol, glycine, skim milk, bovine serum albumin (BSA), and a lyophilization buffer, or any combination thereof.

72. The method of any one of claims 66-71, wherein the Paenibacillus strain is heat-stable.

73. The method of any one of claims 66-72, wherein reducing the risk of, or preventing, or treating an aquaculture pathogen infection in an aquaculture environment comprises reducing or inhibiting growth of one or more pathogens selected from the group consisting of Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum, or any combination thereof.

74. The method of any one of claims 66-73, wherein the aquaculture pathogen infection comprises one or more conditions selected from the group consisting of acute hepatopancreatic necrosis disease (AHPND), early mortality syndrome (EMS), white feces disease (WFD), white spot syndrome, piscirickettiosis, photobacteriosis, meningitis, bacterial kidney disease (BKD), tenacibaculosis, white ulcer disease, columnaris disease, furunculosis, vibriosis, and bacterial cold water disease (BCWD).

75. The method of any one of claims 66-74, wherein the subject is an aquaculture animal.

76. The method of claim 75, wherein the aquaculture animal is a crustacean or cultured fish.

77. The method of claim 76, wherein the aquaculture animal is a crustacean.

78. The method of claim 77, wherein the crustacean is shrimp.

79. The method of 77, wherein the pathogen is Vibrio parahaemolyticus.

80. The method of claim 78 or claim 79, wherein the aquaculture pathogen infection is acute hepatopancreatic necrosis disease (AHPND).

81. The method of any one of claims 66-80, wherein the Paenibacillus strain is administered orally to the subject.

82. The method of any one of claims 66-81, wherein the Paenibacillus strain is administered to the subject as food or a feed additive.

83. The method of any one of claims 66-81, wherein the Paenibacillus strain is administered to the subject as an aquaculture food or an aquaculture feed additive.

84. The method of claim 83, wherein the aquaculture food comprises 1%, 5%, or 10% (w/w) Paenibacillus strain.

85. The method of claim 84, wherein the Paenibacillus strain is a lyophilized bacterial fermentation.

86. A method for improving the growth of an aquaculture animal, comprising administering to the subject an isolated Paenibacillus strain PR-D9 deposited with the Agricultural Research Culture Collection (NRRL—Northern Regional Research Laboratory) under NRRL Accession Number B-68028 or a variant strain thereof, wherein the growth of the aquaculture animal is increased as compared to an untreated control.

87. The method of claim 86, wherein the Paenibacillus strain is formulated as a composition, wherein the composition comprises: (i) the Paenibacillus strain; and (ii) an excipient.

88. The method of claim 86 or claim 87, wherein the Paenibacillus strain is lyophilized.

89. The method of claim 88, wherein the lyophilized Paenibacillus strain is a lyophilized bacterial fermentation (LBF).

90. The method of claim 86 or claim 87, wherein the Paenibacillus strain is spray dried.

91. The method of any one of claims 87-90, wherein the excipient comprises one or more lyoprotectants selected from the group consisting of mannitol, trehalose, sucrose, glycerol, glycine, skim milk, bovine serum albumin (BSA), and a lyophilization buffer, or any combination thereof.

92. The method of any one of claims 86-91, wherein the Paenibacillus strain is heat-stable.

93. The method of any one of claims 86-92, wherein reducing the risk of, or preventing, or treating an aquaculture pathogen infection in an aquaculture environment comprises reducing or inhibiting growth of one or more pathogens selected from the group consisting of Vibrio parahaemolyticus, Whitespot Syndrome Virus, Aeromonas salmonicida, Flavobacterium psychrophilium, Photobacterium damselae piscida, Vibrio anguillarum, Yersinia ruckeri, Streptococcus agalactiae, Streptococcus iniae, Piscrickettsia salmonis, Saprolegnia parasitica, Tenacibaculum maritimum, Moritella viscosa, Flavobacterium columnare, and Renibacterium salmoninarum, or any combination thereof.

94. The method of any one of claims 86-93, wherein the aquaculture pathogen infection comprises one or more conditions selected from the group consisting of acute hepatopancreatic necrosis disease (AHPND), early mortality syndrome (EMS), white feces disease (WFD), white spot syndrome, piscirickettiosis, photobacteriosis, meningitis, bacterial kidney disease (BKD), tenacibaculosis, white ulcer disease, columnaris disease, furunculosis, vibriosis, and bacterial cold water disease (BCWD).

95. The method of any one of claims 86-94, wherein the subject is an aquaculture animal.

96. The method of claim 95, wherein the aquaculture animal is a crustacean or cultured fish.

97. The method of claim 96, wherein the aquaculture animal is a crustacean.

98. The method of claim 97, wherein the crustacean is shrimp.

99. The method of claim 97, wherein the pathogen is Vibrio parahaemolyticus.

100. The method of claim 98 or claim 99, wherein the aquaculture pathogen infection is acute hepatopancreatic necrosis disease (AHPND).

101. The method of any one of claims 86-100, wherein the Paenibacillus strain is administered orally to the subject.

102. The method of any one of claims 86-101, wherein the Paenibacillus strain is administered to the subject as food or a feed additive.

103. The method of any one of claims 86-101, wherein the Paenibacillus strain is administered to the subject as an aquaculture food or an aquaculture feed additive.

104. The method of claim 103, wherein the aquaculture food comprises 1%, 5%, or 10% (w/w) Paenibacillus strain.

105. The method of claim 104, wherein the Paenibacillus strain is a lyophilized bacterial fermentation.

106. A method for improving the nutritional availability of soy protein to an aquaculture animal, the method comprising administering an isolated Paenibacillus strain PR-D9 deposited with the Agricultural Research Culture Collection (NRRL—Northern Regional Research Laboratory) under NRRL Accession Number B-68028 or a variant strain thereof to the aquaculture animal.

107. The method of claim 106, wherein the Paenibacillus strain is formulated as a composition, wherein the composition comprises: (i) the Paenibacillus strain; and (ii) an excipient.

108. The method of claim 106 or claim 107, wherein the Paenibacillus strain is lyophilized.

109. The method of claim 108, wherein the lyophilized Paenibacillus strain is a lyophilized bacterial fermentation (LBF).

110. The method of claim 106 or claim 107, wherein the Paenibacillus strain is spray dried.

111. The method of any one of claims 107-110, wherein the excipient comprises one or more lyoprotectants selected from the group consisting of mannitol, trehalose, sucrose, glycerol, glycine, skim milk, bovine serum albumin (BSA), and a lyophilization buffer, or any combination thereof.

112. The method of any one of claims 106-111, wherein the Paenibacillus strain is heat-stable.

113. The method of any one of claims 106-112, wherein the isolated Paenibacillus strain is administered separately, sequentially, or simultaneously with an aquaculture food.

114. The method of any one of claims 106-113, wherein the isolated Paenibacillus strain is administered to the subject as an aquaculture food or an aquaculture feed additive.
